(19) United States
(12) Patent Application Publication
(10) Pub. No.: US 2018/0363069 A1
(43) Pub. Date: Dec. 20, 2018

(54) METHODS FOR IDENTIFICATION OF NOVEL GENES FOR MODULATING PLANT AGRONOMIC TRAITS

(71) Applicants: PIONEER HI-BRED INTERNATIONAL, INC., JOHNSTON, IA (US); E. I. DU PONT DE NEMOURS AND COMPANY, WILMINGTON, DE (US)

(72) Inventors: SONAL BAKIWALA, JAIPUR (IN); DEBASIS DAN, HYDERABAD (IN); KRUPA DESHMUKH, HYDERABAD (IN); MARY J. FRANK, DES MOINES, IA (US); NANDINI KRISHNAMURTHY, GRIMES, IA (US); BINDU ANDREUZZA, HYDERABAD (IN); ROBERT WAYNE WILLIAMS, JOHNSTON, IA (US); SANGEETA AGARWAL, NEW DELHI (IN)

(73) Assignees: PIONEER HI-BRED INTERNATIONAL, INC., JOHNSTON, IA (US); E. I. DU PONT DE NEMOURS AND COMPANY, WILMINGTON, DE (US)

(21) Appl. No.: 16/063,311

(22) PCT Filed: Dec. 16, 2016
(86) PCT No.: PCT/US16/67207

§ 371 Date: Jun. 18, 2018

Related U.S. Application Data
(60) Provisional application No. 62/269,166, filed on Dec. 18, 2015.

(51) Int. Cl. C12Q 1/6895; G06F 19/20; G06F 19/22; C12N 15/82

(52) U.S. Cl. CPC C12Q 1/6895; G06F 19/20; C12N 15/8201; C12N 15/8271; C12Q 2600/13; G06F 19/22

(57) ABSTRACT

Methods and compositions for identifying novel genes useful for modulating desired agronomic traits in plants are presented herein. The present disclosure relates to methods for identifying line-specific and cluster-specific genes from plants that show perturbation of expression in response to perturbation of expression of a primary gene, and the perturbation of expression of the line-specific or cluster-specific gene confers alterations in agronomic characteristics upon the plant.

### CROSS-REFERENCE TO RELATED APPLICATIONS

This application claims priority to U.S. provisional patent application Ser. No. 62/269166 filed Dec. 18, 2015, herein incorporated by reference in its entirety.

### FIELD

The field relates to plant molecular biology and, in particular, relates to identifying novel genes for modulating important agronomic traits using gene expression information.

### BACKGROUND

Identification of genes with roles in modulating desirable agronomic characteristics in crop plants has high agronomic importance. Desirable agronomic characteristics include traits such as resistance to environmental stresses, increasing crop yield or productivity, and increasing stay-green phenotype. Gene expression analysis can be low-throughput or high-throughput methods. Although large amounts of information for gene expression is available for plants, there is a need to utilize this data for studying genotype-trait relationships and for discovering novel genes and pathways affecting such agronomic traits.

Resistance to abiotic stress and plant yield are typically associated with multigenic traits, making them more complex traits to study. Changes in gene expression that are associated with stress tolerance and increase in plant yield can be complex, and developing methods of identifying the relevant genes from the available gene expression data is a key requirement for increasing plant productivity.

Abiotic stress is also the primary cause of crop loss worldwide, causing average yield losses of more than 50% for major crops (Boyer, J. S. (1982) Science 218:443-448; Bray, E. A. et al. (2000) In Biochemistry and Molecular Biology of Plants, Edited by Buchannan, B. B. et al., Amer. Soc. Plant Biol., pp. 1158-1203). Among the various abiotic stresses, drought and low nitrogen stress are two of the major factors that limit crop productivity worldwide. Understanding of the basic biochemical and molecular mechanism for drought stress perception, transduction and tolerance is a major challenge in biology. Reviews on the molecular mechanisms of abiotic stress responses and the genetic regulatory networks of drought stress tolerance have been published (Valliyodan, B., and Nguyen, H. T., (2006) Curr. Opin. Plant Biol. 9:189-195; Wang, W., et al. (2003) Planta 218:1-14); Vinocur, B., and Altman, A. (2005) Curr. Opin. Biotechnol. 16:123-132; Chaves, M. M., and Oliveira, M. M. (2004) J. Exp. Bot. 55:2365-2384; Shinozaki, K., et al. (2003) Curr. Opin. Plant Biol. 6:410-417; Yamaguchi-Shinozaki, K., and Shinozaki, K. (2005) Trends Plant Sci. 10:88-94, Gallais et al., J. Exp. Bot. 55(396):295-306 (2004)).

### SUMMARY

The present disclosure includes:

A method of identifying at least one line-specific gene from a plurality of plants, wherein all plants in the plurality of plants exhibit alteration in at least one first agronomic characteristic, and wherein the alteration in the at least one first agronomic characteristic in each plant in the plurality of plants is due to perturbation of expression of a different primary gene, when compared to a control plant that does not show the alteration in the at least one first agronomic characteristic, the method comprising the steps of: (a) analyzing gene expression in each plant in the plurality of plants to identify genes that show perturbation of expression when compared to a control plant; (b) comparing gene expression data from a first plant in the plurality of plants to gene expression data from other plants in the plurality of plants to identify at least one line-specific gene from the first plant, wherein the at least one line-specific gene shows perturbation of expression in the first plant, and wherein the at least one line-specific gene from the first plant does not show the same perturbation of expression in any of the other plants in the plurality of plants. In the present disclosure, the method of identifying a line-specific gene further may comprise the step of selecting a line-specific gene, wherein the line-specific gene confers upon a plant an alteration in the at least one first agronomic characteristic, wherein the plant shows a perturbation in expression of the line-specific gene when compared to a control plant.

The perturbation of expression in the line-specific gene may be used as marker for the first plant to distinguish the first plant from the rest of the plants in the plurality of plants. The perturbation of expression of the primary gene may be overexpression. The perturbation of expression of the primary gene may be downregulation.

The at least one step of the method may be done computationally. Step (b) may be done by using a machine learning algorithm. The order of partial correlation between said first gene with perturbed expression in the first plant and said line-specific gene identified from the first plant in the plurality of plants may be not more than two. The term “correlation”, as used herein, relates to any of a class of statistical relationships involving dependence, wherein dependence is defined as any statistical relationship between two random variables or two sets of data. As used herein “partial correlation” measures the correlation between two variables after their linear dependence on other variables is removed. It can distinguish between direct and indirect associations (Zuo et al (2014) Methods 69: 266-273.

In the present disclosure, the order of partial correlation between the primary gene and the line-specific gene may be not more than two. In the present disclosure, the correlation between the primary gene and the line-specific gene may be zero order partial correlation, first order partial correlation, or second order partial correlation.

The current disclosure includes a method of identifying at least one cluster specific gene from a plurality of plants, wherein all plants in the plurality of plants exhibit an alteration in at least one first agronomic characteristic, the method comprising the steps of: (a) identifying at least one first cluster of plants and at least one second cluster of plants from the plurality of plants, wherein clustering is done on the basis of criteria selected from the group consisting of: (i) alteration in at least one second agronomic characteristic in all the plants of a cluster; (ii) similarity in gene expression profile between the plants of a cluster as determined by the distance metric with a cluster bootstrap confidence value of at least 50%; in the present disclosure, the bootstrap confidence value for the plants in the same cluster is at least 60%. (iii) perturbed expression of polypeptides from the same gene family in all plants from the same cluster; (b) analyzing gene expression in plants from the at least one first cluster of plants and the at least one second cluster of plants; (c) comparing the gene expression data from the at least one first cluster of plants to the gene expression data from the at least one second cluster of plants; (d) identifying at least one cluster-specific gene that shows perturbed expression in at least 80% of the plants from the at least one first cluster of plants, and perturbed in not more than 20% of the plants from the at least one second cluster of plants. The cluster specific gene may show perturbed expression in not more than 10% of the plants from the at least one second cluster of plants.

The method of identifying a cluster-specific gene further may comprise the step of selecting a cluster-specific gene, wherein the cluster-specific gene confers upon a plant an alteration in the at least one first agronomic characteristic, wherein the plant shows a perturbation of expression of the cluster-specific gene when compared to a control plant.

In a method of identifying a cluster-specific gene from a plurality of plants, the alteration in the at least one first agronomic characteristic in each plant in the plurality of plants may be due to perturbation of expression of a different gene. The alteration in the at least one first agronomic characteristic in each plant in the plurality of plants may be due to perturbation of expression of the same gene. The at least one step of the method may be done computationally. The at least one step of the method that is done computationally may be done by using a machine learning algorithm.

The step for analyzing gene expression data in any of the methods for identifying at least one line-specific gene or for identifying at least one cluster-specific gene may be done in specific tissues. Said line-specific gene or cluster-specific gene may be identified from the plurality of plants that shows perturbation of expression in all the tissues analyzed for gene expression.

Each plant in the plurality of plants may comprise a recombinant construct comprising a polynucleotide sequence that comprises the coding region of the primary gene operably linked to at least one heterologous regulatory element. “Heterologous” with respect to sequence means a sequence that originates from a foreign species, or, if from the same species, is substantially modified from its native form in composition and/or genomic locus by deliberate human intervention.

The plurality of plants may comprise at least two plants. The plurality of plants may comprise at least 10 plants. All plants in the plurality of plants may exhibit alteration in at least one first agronomic characteristic, and wherein said all plants in said plurality of plants exhibit alteration in the same at least one first agronomic characteristic. All plants in the plurality of plants may exhibit alteration in at least one first agronomic characteristic, wherein said all plants in said plurality of plants do not exhibit alteration in the same at least one first agronomic characteristic.

The current disclosure includes a polynucleotide encoding a transcript of a line-specific or cluster-specific gene identified by any of the methods disclosed herein, wherein said polynucleotide, upon perturbation of expression in a plant, confers upon said plant at least one phenotype, wherein the phenotype is selected from the group consisting of: increased yield, increased productivity and increased stress resistance, when compared to a control plant. The current disclosure includes a recombinant DNA construct comprising the polynucleotide, wherein the polynucleotide is operably linked to a heterologous regulatory element, and wherein said recombinant DNA construct confers upon a plant comprising said recombinant DNA construct at least one phenotype, wherein the phenotype is selected from the group consisting of: increased yield, increased productivity and increased stress resistance, when compared to a control plant. The current disclosure includes a plant comprising the recombinant DNA construct comprising the polynucleotide encoding the transcript of a line-specific or cluster-specific gene, wherein the plant exhibits alteration in at least one phenotype, wherein the phenotype is selected from the group consisting of: increased yield, increased productivity and increased stress resistance, when compared to a control plant.

The current disclosure includes the use of the polynucleotide or the recombinant DNA construct disclosed herein, to produce a plant that exhibits alteration in at least one phenotype, wherein the phenotype is selected from the group consisting of: increased yield, increased productivity and increased stress resistance, when compared to a control plant.

The current disclosure includes the use of the at least one line specific gene and/or the at least one cluster specific gene identified by the methods disclosed herein, to identify at least one other line-specific gene and/or cluster-specific gene.

### BRIEF DESCRIPTION OF THE DRAWINGS

The disclosure can be more fully understood from the following detailed description and the accompanying drawings which form a part of this application.

FIG. 1 shows clustering of the 48 transgenic lines based on gene expression data in root tissue, by Hclust method. The oval marks a robust cluster that was identified; the cluster is made of three transgenic plants, comprising transgenes AT7, AT8 and AT9. The x-axis shows the validation status of the different transgenic lines (AT1, AT2 . . . ) in either low nitrogen stress assay (LN); root architecture assay (RA assay); Nitrogen uptake (NU); and genes that validated in RA as well as LN assay are marked as T. Y-axis shows the clustering height that is the value of the criterion associated with the clustering method for the particular agglomeration.

FIG. 2 shows clustering of the 48 transgenic lines based on gene expression data in shoot tissue, by Hclust method. The oval marks a robust cluster that was identified; the cluster is made of three transgenic plants, comprising transgenes AT7, AT8 and AT9. The x-axis shows the validation status of the different transgenic lines (AT1, AT2 . . . ) in either low nitrogen stress assay (LN); root architecture assay (RA assay); Nitrogen uptake (NU); and genes that validated in RA as well as LN assay are marked as T. Y-axis shows the clustering height that is the value of the criterion associated with the clustering method for the particular agglomeration.

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

The present disclosure provides methods and compositions for identifying at least one line-specific gene and/or cluster-specific gene from a plurality of plants that when expressed confers upon a plant an alteration in at least one agronomic characteristic. Without wishing to be bound by this theory, it is believed that use of the methods and compositions described herein results in the identification of line-specific genes that are less random and have higher confidence values associated with the results. For instance, line-specific genes identified through these processes have high validation rates, e.g. are more likely to exhibit a same or similar phenotype/trait of the agronomic characteristic of the primary gene, when expressed and tested in additional assays and various conditions. See, for example, Example 5. Without wishing to be bound by this theory, it is believed that use of the line-specific genes identified by the methods described herein in methods of identifying cluster-specific genes is believed to improve the confidence of these results and have high validation rates as well. See, for example, Example 5. The current disclosure includes a method for identifying line-specific genes and cluster-specific genes, wherein each line-specific gene and cluster-specific gene is associated with a particular biological pathway. The line-specific gene and cluster-specific gene may be used as markers for distinguishing a plant or cluster of plants respectively, from other plants or cluster of plants, in that particular plurality of plants.

As used herein, the term “line-specific gene” or “line-specific marker” (LSM) are used interchangeably herein, and refer to a gene that shows perturbed expression in one plant from a group or plurality of plants, but does not show the same perturbation of expression in other plants from that group or plurality of plants. As used herein, the term “marker” gene is defined as any gene that may be used to differentiate a plant from other plants in the same plurality of plants. In the context of the current disclosure the marker gene is used to distinguish the plant from other plants in the same plurality of plants, or cluster of plants from other cluster of plants in the same plurality of plants.

The term “plurality” of plants refers to a group or population of plants with a defined number of plants. For the purposes of the current disclosure, the plurality of plants used for the methods disclosed herein may comprise of any number of plants, and the selection of “plurality of plants” for the purposes of the current disclosure is not limited by the number of plants in the plurality of plants. The plurality of plants may comprise of at least two, at least there, at least four, at least five, at least six, at least seven, at least eight, at least nine, or at least ten or more plants.

The present disclosure includes methods of identifying at least one line-specific gene from a plurality of plants, wherein all plants in the plurality of plants exhibit an alteration in at least one first agronomic characteristic.

As used herein, “agronomic characteristic” is a measurable parameter including but not limited to, abiotic stress tolerance, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, abiotic stress tolerance, biotic stress tolerance, drought tolerance, nitrogen uptake, root lodging, harvest index, stalk lodging, plant height, ear height, ear length, leaf number, tiller number, growth rate, first pollen shed time, silk length, first silk emergence time, anthesis silking interval (ASI), stalk diameter, root architecture, staygreen, relative water content, water use, water use efficiency; dry weight of either main plant, tillers, primary ear, main plant and tillers or cobs; rows of kernels, total plant weight, kernel weight, kernel number, salt tolerance, chlorophyll content, flavonol content, number of yellow leaves, leaf appearance rate, grain moisture content, early seedling vigor and seedling emergence under low temperature stress. These agronomic characteristics maybe measured at any stage of the plant development. One or more of these agronomic characteristics may be measured under stress or non-stress conditions, and may show alteration on overexpression of the polynucleotides or recombinant constructs disclosed herein.

As described herein the alteration in an “agronomic characteristic” may be a change in a plant in any of the characteristics described above or elsewhere herein. In some embodiments, alter, altering or alteration in an “agronomic characteristic” refers to any kind of change, for example, increase or decrease in the nature or intensity of an agronomic characteristic displayed by the plant, for example, under a particular set of conditions or environmental factors, including assay, controlled environment, greenhouse or field conditions as compared to a control. In some examples, the “agronomic characteristic” of one plant will be compared to the “agronomic characteristic” of an appropriate plant, for example, a control plant not exhibiting perturbation of expression of a primary gene, and/or a line-specific gene, and/or a cluster-specific gene or having an alteration in the at least one first agronomic characteristic or wild type plant. In some examples, the change is statistically significant. In some embodiments, the plurality of plants exhibit an alteration in at least one first agronomic characteristic so that the plurality of plants considered in the analysis have the same effect on an agronomic characteristic or trait of interest. For example, in reference to drought tolerance, all the primary genes considered may improve drought tolerance in contrast to a combination of genes some of which improve and some of them sensitize the plants towards drought tolerance.

The change in an agronomic characteristic is determined with respect to a control or wild-type plant. Many of the agronomic characteristics and the assays by which the alterations in which agronomic characteristics can be measured have been described in US patent publication Nos. US2014304854, US2009011516. In some instances, the agronomic characteristics for the same trait can be measured in different ways or using different assays. For example, drought stress resistance can be measured by an increase in triple stress resistance and an increase resistance observed in in soil drought assay and could be counted as two distinct agronomic characteristics for the purposes of the current disclosure, i.e. a first and second agronomic characteristics.

An alteration in an agronomic characteristic in a plant may be measured by any of the methods that are well-known in prior art. Many of these methods have been described in US2014304854, US2009011516. The alteration in the at least one first agronomic characteristic in each plant in the plurality of plants is due to perturbation of expression of a different primary gene. The term “primary gene” as used herein refers to a gene that is responsible for the alteration in the at least one first agronomic characteristic in the plants in the plurality or group of plants used for identifying line-specific gene or cluster specific gene. In some, examples, the primary gene is different from the line-specific or cluster-specific gene. The more than one line-specific gene may be identified from the first plant in the plurality of plants, wherein the first plant exhibits an alteration in at least one first agronomic characteristic due to perturbation of expression of a primary gene. The primary gene and the at least one line-specific gene showing perturbation of expression in the first plant, may be in the same biological pathway. The line-specific gene may be close to the primary gene in the pathway. For example, the line-specific gene may be linked directly or indirectly to the primary gene to affect the referred pathway. Accordingly, a plurality or group of plants used for identifying a line-specific gene can comprise plants that show an alteration in at least one first agronomic trait or characteristic, as a result of perturbation of expression of a different primary gene in each plant. The plant may be a hybrid plant or an inbred plant. Any plant having an alteration in at least one first agronomic trait or characteristic, as a result of perturbation of expression of a different primary gene in each plant may be used in the methods described herein, including but not limited to transgenics, inbreds, hybrids, genome edited, and non-transformed plants. This also includes plants that have been treated with a mutagen, such as ethyl methanesulfonate (EMS) and the like.

In some examples, the alteration in the at least one first agronomic characteristic in each plant in the plurality of plants is determined as compared to a control plant that does not show the alteration in the at least one first agronomic characteristic.

The expression of the primary gene encoded by an endogenous locus in a plant may be perturbed, as compared to a control plant, from mutagenesis techniques or genome editing approaches described herein and available to one of ordinary skill in the art. The expression of the primary gene encoded by an endogenous locus in a plant may be perturbed, when compared to a control plant, due to allelic variation.

The perturbation of expression of the line-specific gene is due to perturbation of expression of the primary gene and/or is due to the alteration in the at least one first agronomic characteristic.

As used herein the terms “perturbation of expression of a gene” or “gene perturbation” are used interchangeably herein, and refer to the change in expression levels of a gene, when measured relative to a control or wild-type plant. In other examples, the plurality or population of plants used for the methods disclosed herein do not include any control or wild-type plants. For the purposes of the current disclosure, each plant in the plurality of plants exhibits alteration in at least one first agronomic characteristic, and perturbed expression of at least one primary gene, when compared to a control or wild-type plant. So, each plant in a plurality of plants used herein for identifying an LSM and/or a CSM, is preselected by comparison to a control plant for perturbation of a primary gene, and for alteration of at least one first agronomic characteristic.

This also entails that for purposes of the current disclosure, no comparison of gene expression differences is made to a control or wild-type plant for the identification of an LSM and/or CSM, wherein the control plant doesn't exhibit perturbation in expression of the primary gene and also does not exhibit an alteration in the at least one first agronomic characteristic.

The perturbation or change in levels of expression can be either lowering or suppression of gene expression levels, or an increase in expression or overexpression of the gene. The perturbation of expression of the primary gene, when compared to a control plant, may be achieved using any suitable approach or technique, including transgenic or non-transgenic approaches. In some instances, the primary gene may be overexpressed in a plant or downregulated in a plant. The primary gene may be an endogenous gene or heterologous with respect to the plant genome. The perturbation of expression of the primary genes in all plants in one plurality of plants may be overexpression. The perturbation of expression of the primary genes in all plants in one plurality of plants may be downregulation. The perturbation of expression of the primary genes in some plants in one plurality of plants may be downregulation, and may be overexpression in other plants of the same plurality of plants. When two genes are referred to as having a “perturbation of expression in the same direction”, or the “same perturbation of expression”, as used herein, it means that they both have either suppression of expression levels or both have increase in expression levels. When one or more genes are referred to as having a “perturbation of expression in opposite or different direction”, as used herein, it refers to the fact that the perturbation of expression is overexpression for one gene, and suppression of expression for the other gene. A single gene may have perturbation of expression in the “same direction” or “different direction” in two different tissues or plants or plant lines.

Any kind of changes in the expression of a “primary gene” may lead to alteration in the at least one first agronomic characteristic. The primary gene may have perturbation of expression in at least one tissue of the plant, or during at least one condition of environmental stress, or both. The change or perturbation of expression in a primary gene may be overexpression or suppression.

The perturbation in expression of a gene may be due to any reason, many of which are well known in the art. The strength of a promoter is well known as major factor regulating gene expression. A strong, constitutive promoter can drive high levels of gene expression in most of the tissues. Many of the promoters that can be used for the methods and compositions of this disclosure have been discussed elsewhere in this specification. Mutations or changes in promoters can lead to changes in gene expression. Other regulatory elements such as enhancers, introns, also regulate gene expression, and any changes in these elements such as sequence changes, or removing or adding copies can lead to changes in gene expression. Mutations can include insertions, deletions, nucleotide substitutions, and combinations thereof. Changes in gene expression can also be due to epigenetic changes.

In methods of the current disclosure, the expression of the primary gene may be modulated by transgenic approaches. The transgenic modifications may be overexpression of a transgene or suppression of gene expression by transgenic techniques.

The present disclosure includes methods wherein each plant in the plurality of plants comprises a recombinant construct that comprises a polynucleotide sequence, wherein the polynucleotide sequence comprises the coding region of the primary gene, and wherein the polynucleotide is operably linked to at least one heterologous regulatory element.

In the present disclosure, the perturbation in expression of the primary gene may be due to non-transgenic approaches. In the present disclosure, the primary gene may be an endogenous gene, and is located at a particular genetic locus, and the perturbation in expression which leads to the alteration in the at least one first agronomic characteristic may be due to “mutation or alteration in the chromosomal locus”, or due to an epigenetic change at the endogenous locus.

As used herein, the phrases “mutated chromosomal loci”, “mutated chromosomal locus”), “chromosomal mutations” and “chromosomal mutation” refer to portions of a chromosome that have undergone a heritable genetic change in a nucleotide sequence relative to the nucleotide sequence in the corresponding parental chromosomal loci. Mutated chromosomal loci comprise mutations that include, but are not limited to, nucleotide sequence inversions, insertions, deletions, substitutions, site-specific mutations, or combinations thereof. In the present disclosure, the mutated chromosomal loci can comprise mutations that are irreversible or reversible. Reversible mutations in the chromosome can include, but are not limited to, insertions of transposable elements, defective transposable elements, and certain inversions. Mutations in chromosomal or genetic loci can include insertions, deletions, nucleotide substitutions, and combinations thereof.

Mutations in the endogenous gene may be caused by insertional mutagenesis including but not limited to transposon mutagenesis, or it may be caused by zinc finger nuclease, Transcription Activator-Like Effector Nuclease (TALEN), CRISPR or meganuclease (Burgess D J (2013) Nat Rev Genet 14:80; PCT publication No. WO2014/127287; PCT publication No. WO2014127287; US Patent Publication No. US20140087426).

Methods and techniques to modify or alter primary genes, line-specific genes and cluster-specific genes are available. In some examples, this includes altering the host plant native DNA sequence or a pre-existing recombinant sequence including regulatory elements, coding and/or non-coding sequences. These methods are also useful in targeting nucleic acids to pre-engineered target recognition sequences in the genome. As an example, a modified cell or plant may be generated using “custom” or engineered endonucleases such as meganucleases produced to modify plant genomes (see e.g., WO 2009/114321; Gao et al. (2010) Plant Journal 1:176-187). Another site-directed engineering is through the use of zinc finger domain recognition coupled with the restriction properties of restriction enzyme. See e.g., Urnov, et al., (2010) Nat Rev Genet. 11(9):636-46; Shukla, et al., (2009) Nature 459 (7245):437-41. A transcription activator-like (TAL) effector-DNA modifying enzyme (TALE or TALEN) is also used to engineer changes in plant genome. See e.g., US20110145940, Cermak et al., (2011) Nucleic Acids Res. 39(12) and Boch et al., (2009), Science 326(5959): 1509-12. Site-specific modification of plant genomes can also be performed using the bacterial type II CRISPR (clustered regularly interspaced short palindromic repeats)/Cas (CRISPR-associated) system. See e.g., Belhaj et al., (2013), Plant Methods 9: 39; The Cas9/guide RNA-based system allows targeted cleavage of genomic DNA guided by a customizable small noncoding RNA in plants (see e.g., WO 2015026883A1).

In an embodiment, through genome editing approaches described herein and those available to one of ordinary skill in the art, regulatory elements, coding, or non-coding sequences of endogenous genes, such as native genes, of pre-existing recombinant sequences in the plant genome or of recombinant DNA constructs can be engineered to perturb the expression of one or more primary genes, line-specific genes, cluster-specific genes, including those line-specific genes or cluster-specific genes identified by the methods disclosed herein.

Mutagenic techniques may also be employed to introduce mutations into a plant genome that could lead to perturbation of expression of the primary gene. Methods for introducing genetic mutations into plant genes and selecting plants with desired traits are well known. For instance, seeds or other plant material can be treated with a mutagenic chemical substance, according to standard techniques. Such chemical substances include, but are not limited to, the following: diethyl sulfate, ethylene imine, and N-nitroso-N-ethylurea. Alternatively, ionizing radiation from sources such as X-rays or gamma rays can be used.

“TILLING” or “Targeting Induced Local Lesions IN Genomics” refers to a mutagenesis technology useful to generate and/or identify, and to eventually isolate mutagenised variants of a particular nucleic acid with modulated expression and/or activity (McCallum et al., (2000), Plant Physiology 123:439-442; McCallum et al., (2000) Nature Biotechnology 18:455-457; and, Colbert et al., (2001) Plant Physiology 126:480-484). TILLING also allows selection of plants carrying mutant variants. These mutant variants may exhibit modified expression, either in strength or in location or in timing (if the mutations affect the promoter for example).

As used herein, the phrases “epigenetic modifications” or “epigenetic modification” refer to heritable and reversible epigenetic changes that include, but are not limited to, methylation of chromosomal DNA, and in particular, methylation of cytosine residues to 5-methylcytosine residues. Changes in DNA methylation of a region are often associated with changes in sRNA levels with homology to the region and are derived from the region.

As used herein, the phrases “suppression”, “downregulation” or “suppressing expression” of a gene refer to any genetic, nucleic acid, nucleic acid analog, environmental manipulation, grafting, transient or stably transformed methods of any of the aforementioned methods, or chemical treatment that provides for decreased levels of gene expression, in a plant or plant cell relative to the levels of gene expression that occur in an otherwise isogenic plant or plant cell that had not been subjected to this genetic or environmental manipulation (control plant).

Suppression techniques by transgenic approaches that can result in decreased expression of a gene by a variety of mechanisms include, but are not limited to, dominant-negative mutants, small inhibitory RNA (siRNA), microRNA (miRNA), co-suppressing sense RNA, ribozymes and/or anti-sense RNA. U.S. patents incorporated herein by reference in their entireties that describe suppression of endogenous plant genes by transgenes include U.S. Pat. No. 7,109,393, U.S. Pat. No. 5,231,020 and U.S. Pat. No. 5,283,184 (co-suppression methods); and U.S. Pat. No. 5,107,065 and U.S. Pat. No. 5,759,829 (antisense methods). Transgenes specifically designed to produce double-stranded RNA (dsRNA) molecules with homology to the endogenous gene of a chromosomal locus can also be used to decrease expression of an endogenous gene. The sense strand sequences of the dsRNA can be separated from the antisense sequences by a spacer sequence, preferably one that promotes the formation of a dsRNA (double-stranded RNA) molecule. Wesley et al., Plant J., 27(6):581-90 (2001), Hamilton et al., Plant J., 15:737-746 (1998), U.S. Patent Application Nos. 20050164394, 20050160490, and 20040231016, each of which is incorporated herein by reference in their entirety.

“Suppression DNA construct” is a recombinant DNA construct which when transformed or stably integrated into the genome of the plant, results in “silencing” of a target gene in the plant. The target gene may be endogenous or transgenic to the plant. “Silencing,” as used herein with respect to the target gene, refers generally to the suppression of levels of mRNA or protein/enzyme expressed by the target gene, and/or the level of the enzyme activity or protein functionality. The terms “suppression”, “downregulation” “suppressing” and “silencing”, used interchangeably herein, include lowering, reducing, declining, decreasing, inhibiting, eliminating or preventing. “Silencing” or “gene silencing” does not specify mechanism and is inclusive, and not limited to, anti-sense, cosuppression, viral-suppression, hairpin suppression, stem-loop suppression, RNAi-based approaches, and small RNA-based approaches.

A suppression DNA construct may comprise a region derived from a target gene of interest and may comprise all or part of the nucleic acid sequence of the sense strand (or antisense strand) of the target gene of interest. Depending upon the approach to be utilized, the region may be 100% identical or less than 100% identical to all or part of the sense strand (or antisense strand) of the gene of interest.

A suppression DNA construct may comprise a region derived from a target gene of interest and may comprise all or part of the nucleic acid sequence of the sense strand (or antisense strand) of the target gene of interest. Depending upon the approach to be utilized, the region may be 100% identical or less than 100% identical (e.g., at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical) to all or part of the sense strand (or antisense strand) of the gene of interest.

A suppression DNA construct may comprise 100, 200, 300, 400, 500, 600, 700, 800, 900 or 1000 contiguous nucleotides of the sense strand (or antisense strand) of the gene of interest, and combinations thereof.

Suppression DNA constructs are well-known in the art, are readily constructed once the target gene of interest is selected, and include, without limitation, cosuppression constructs, antisense constructs, viral-suppression constructs, hairpin suppression constructs, stem-loop suppression constructs, double-stranded RNA-producing constructs, and more generally, RNAi (RNA interference) constructs and small RNA constructs such as sRNA (short interfering RNA) constructs and miRNA (microRNA) constructs.

Suppression of gene expression may also be achieved by use of artificial miRNA precursors, ribozyme constructs and gene disruption. A modified plant miRNA precursor may be used, wherein the precursor has been modified to replace the miRNA encoding region with a sequence designed to produce a miRNA directed to the nucleotide sequence of interest. Gene disruption may be achieved by use of transposable elements or by use of chemical agents that cause site-specific mutations.

“Antisense inhibition” generally refers to the production of antisense RNA transcripts capable of suppressing the expression of the target gene or gene product. “Antisense RNA” generally refers to an RNA transcript that is complementary to all or part of a target primary transcript or mRNA and that blocks the expression of a target isolated nucleic acid fragment (U.S. Pat. No. 5,107,065). The complementarity of an antisense RNA may be with any part of the specific gene transcript, i.e., at the 5′ non-coding sequence, 3′ non-coding sequence, introns, or the coding sequence.

“Cosuppression” generally refers to the production of sense RNA transcripts capable of suppressing the expression of the target gene or gene product. “Sense” RNA generally refers to RNA transcript that includes the mRNA and can be translated into protein within a cell or in vitro. Cosuppression constructs in plants have been previously designed by focusing on overexpression of a nucleic acid sequence having homology to a native mRNA, in the sense orientation, which results in the reduction of all RNA having homology to the overexpressed sequence (see Vaucheret et al., Plant J. 16:651-659 (1998); and Gura, Nature 404:804-808 (2000)).

Another variation describes the use of plant viral sequences to direct the suppression of proximal mRNA encoding sequences (PCT Publication No. WO 98/36083 published on Aug. 20, 1998).

RNA interference generally refers to the process of sequence-specific post-transcriptional gene silencing in animals mediated by short interfering RNAs (siRNAs) (Fire et al., Nature 391:806 (1998)). The corresponding process in plants is commonly referred to as post-transcriptional gene silencing (PTGS) or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., Trends Genet. 15:358 (1999)).

Small RNAs play an important role in controlling gene expression. Regulation of many developmental processes, including flowering, is controlled by small RNAs. It is now possible to engineer changes in gene expression of plant genes by using transgenic constructs which produce small RNAs in the plant.

Small RNAs appear to function by base-pairing to complementary RNA or DNA target sequences. When bound to RNA, small RNAs trigger either RNA cleavage or translational inhibition of the target sequence. When bound to DNA target sequences, it is thought that small RNAs can mediate DNA methylation of the target sequence. The consequence of these events, regardless of the specific mechanism, is that gene expression is inhibited.

MicroRNAs (miRNAs) are noncoding RNAs of about 19 to about 24 nucleotides (nt) in length that have been identified in both animals and plants (Lagos-Quintana et al., Science 294:853-858 (2001), Lagos-Quintana et al., Curr. Biol. 12:735-739 (2002); Lau et al., Science 294:858-862 (2001); Lee and Ambros, Science 294:862-864 (2001); Llave et al., Plant Cell 14:1605-1619 (2002); Mourelatos et al., Genes Dev. 16:720-728 (2002); Park et al., Curr. Biol. 12:1484-1495 (2002); Reinhart et al., Genes. Dev. 16:1616-1626 (2002)). They are processed from longer precursor transcripts that range in size from approximately 70 to 200 nt, and these precursor transcripts have the ability to form stable hairpin structures.

MicroRNAs (miRNAs) appear to regulate target genes by binding to complementary sequences located in the transcripts produced by these genes. It seems likely that miRNAs can enter at least two pathways of target gene regulation: (1) translational inhibition; and (2) RNA cleavage. MicroRNAs entering the RNA cleavage pathway are analogous to the 21-25 nt short interfering RNAs (siRNAs) generated during RNA interference (RNAi) in animals and posttranscriptional gene silencing (PTGS) in plants, and likely are incorporated into an RNA-induced silencing complex (RISC) that is similar or identical to that seen for RNAi.

Gene expression data for any of the genes used in the methods and compositions described herein, e.g. primary genes, line-specific genes, or cluster-specific genes, may be collected from samples of any desired plant or tissue, for example, from but not limited to, maize root, maize shoot, maize leaf, maize ear, soy root, soy shoot, or soy leaf tissue. In some examples, the gene expression data is transcriptomics. In some cases, the primary gene is over-expressed or downregulated in a plant compared to the expression of a control plant that doesn't exhibit perturbation in expression of the primary gene and also does not exhibit an alteration in the at least one first agronomic characteristic.

The current disclosure includes the steps of analyzing gene expression and comparing gene expression data between plants or cluster of plants, wherein the comparison is always done between plants that exhibit perturbed expression of at least one primary gene, when compared to a control or wild-type plant. The step of comparing gene expression data from the first plant to the other plants in the plurality of plants may be done manually or computationally or both.

Analysis of gene expression may be done by any method, many of which are well known in the art. Gene expression for a few numbers of genes can be analyzed by well-known procedures such as reverse-transcriptase PCR, Northern blotting, RNase protection assay and differential display technologies. Some variations of the basic RT-PCR techniques such as quantitative PCR (qRT-PCR) and real-time quantitative RT-PCR (qRT-PCR) are also frequently used for gene expression analysis of small to moderate number of genes. qRT-PCR can be done by using many technologies, such as fluorophore technologies, that are well known in art. All these techniques may be used for detecting, quantifying and characterizing RNA species. Transcript profiling, or gene expression analysis at high-throughput mode, can be done for analyzing gene expression using techniques such as microarrays, MPSS (massively parallel signature sequencing), SAGE (Serial analysis of gene expression) and RNA-seq (VanGuilder et al BioTechniques 44:619-626 (2008); Baginsky et al Plant Physiology, February 2010, Vol. 152, pp. 402-410; Rapaport et al (2013) Genome Biology, 14:R95; Ozsolak et al Nature Reviews Genetics 12, 87-98 (February 2011); Tuteja et al (2004) BioEssays 26:916-922; Liang and Pardee (1995) Current Opinion Immun, 7:274-280). If desired, the expression level of each gene may be determined in relation to various features of the expression products of the gene including exons, introns, and protein activity.

Expression levels of at least two genes are measured in each plant belonging to a plurality of plants. Expression of at least 2, at least 10, at least 100, at least 1000 or at least 10000 genes or more is measured in each plant in a plurality of plants, for the purposes of the current disclosure.

The method comparing gene expression data may include the steps of: (a) analyzing gene expression in each plant in the plurality of plants to identify genes that show perturbation of expression when compared to a control plant; (b) comparing gene expression data from a first plant in the plurality of plants to gene expression data from other plants in the plurality of plants to identify at least one line-specific gene from the first plant, wherein the at least one line-specific gene shows perturbation of expression in the first plant, and wherein the at least one line-specific gene from the first plant does not show the same perturbation of expression in any of the other plants in the plurality of plants.

Comparing gene expression data using the datasets generated by using any of the techniques to detect gene expression profiles can be done manually or computationally. Small numbers of gene expression data from small number of samples can be compared with or without computational methods. The step of gene expression data comparison may be done by using a machine learning algorithm. The step of comparing gene expression data may be done by using a pattern-recognition algorithm.

Technologies such as microarray, RNA-seq, SAGE can produce large amounts of data, which can be interpreted by computational methods. The first computational steps of interpretation of gene expression data encompass the pre-processing of the data and the use of statistical tests to detect genes with altered expression. Tools and methods for analysis of gene expression data are well known in art. Tools for network analysis software such as Matlab or R, Genevestigator, MapMan are non-limiting examples (Bassel et al Plant Cell (2012) vol. 24 (10): 3859-3875).

Comparison of gene expression levels and classification of genes depending on expression levels using computational methods can be done using an algorithm. Any suitable procedure can be utilized for processing gene expression measurements or data sets. Non-limiting examples of procedures suitable for use for processing data sets include filtering, normalizing, weighting, monitoring peak heights, monitoring peak areas, monitoring peak edges, determining area ratios, mathematical processing of data, statistical processing of data, application of statistical algorithms, analysis with fixed variables, analysis with optimized variables, plotting data to identify patterns or trends for additional processing, the like and combinations of the foregoing. In some examples, raw gene expression measurements are put through various preprocessing steps that can be done through the application of algorithms designed to normalize and or improve the reliability of the data. The data analysis can require a computer or other device, machine or apparatus for application of the various algorithms described herein due to the large number of individual data points that are processed (Asyali et al Curr. Bioinformatics, 2006, 1, 55-73, Bassel et al Plant Cell October 2012 vol. 24 no. 10 3859-387). Different normalization techniques can be used for the microarray data, and are well known in art (Wilson et al Bioinformatics 2003; 19: 1325-32, Smyth G K and Speed T. Methods 2003; 31: 265-73). In some examples, the data set is normalized. See, for example, Example 1.

The method of identifying a line-specific gene may further comprise the step of selecting a line-specific gene that confers upon a plant an alteration in the at least one first agronomic characteristic, and where the plant shows a perturbation of expression of the line-specific gene when compared to a control plant. The perturbation of expression of the line-specific gene may be responsible for the alteration in the at least one first agronomic characteristic in the plant. The perturbation of expression of a line-specific gene in a plant may confer upon the plant an alteration in at least one agronomic characteristic other than the first agronomic characteristic, e.g. a second agronomic characteristic. Agronomic characteristics are known to those in the art and also described elsewhere herein.

In part, these methods may include using a p-value. For example, in determining what line-specific genes to select, for example, for testing and further evaluation, a p-value cutoff may be used to identify those genes that have differential expression compared to gene expression from a control plant, where the control plant does not exhibit perturbation in expression of the primary gene and also does not exhibit an alteration in the at least one first agronomic characteristic. In some cases, the plant contains a wild-type primary gene that is not perturbed in expression.

A p-value of less than or equal to 0.10, 0.09, 0.08, 0.07, 0.06, 0.05, 0.04, 0.03, 0.02, 0.01 or 0.005 may be used in these methods, for example, using those genes where the expression data had a value less than or equal to a p-value of 0.1. The data from primary genes that have differential expression that meets or is less than a desired determined p-value, for example, 0.1 or 0.01, may then be used for the identification of the line-specific genes.

In cases where the data is highly unbalanced, for example, where there are less number of samples in one class versus too many samples in the class that it is being compared to, the data may be put into different classes and the same number of data is taken from both classes so that the number of variables randomly sampled is reduced. See, for example, Example 1.

One or more algorithms may be used to further process the data, including data that made the p-value cutoff (below the determined desired p-value), including but not limited to machine learning algorithms. A “machine learning algorithm” can refer to a computational-based prediction methodology, also known to persons skilled in the art as a “classifier”, employed for characterizing a gene expression profile. The signals corresponding to certain expression levels, which can be obtained by, e.g., microarray-based hybridization assays, can be subjected to the algorithm in order to classify the expression profile. Supervised learning can involve “training” a classifier to recognize the distinctions among classes and then “testing” the accuracy of the classifier on an independent test set. For new, unknown samples, the classifier can be used to predict the class in which the samples belong (PCT publication No. WO2014151764, Asyali et al Curr. Bioinformatics, 2006, 1, 55-73, Greene et al J. Cell. Physiol. 229: 1896-1900, 2014, Maetschke et al Briefings in Bioinformatics. 2014; 15(2):195-211).

Any machine learning algorithm can be used in the methods of the current disclosure. Some examples of the machine learning algorithms include, but are not limited to, Support Vector Machine algorithms, Random Forest, Neural Network algorithms, Naïve Bayesian algorithms, Partial Least square algorithm, and combinations thereof (Kursa M. B. BMC Bioinformatics 2014, 15:8; Greene et al J. Cell. Physiol. 229: 1896-1900, 2014). As is well known in art, machine learning methods can be run in unsupervised, semi-supervised and supervised modes. Unsupervised methods do not use any data to adjust internal parameters. Supervised methods, on the other hand, exploit all data to optimize parameters such as weights or thresholds. Semi-supervised methods use only part of the data for parameter optimization.

The primary genes may be ranked, scored or otherwise assigned a value for example, an importance value, using any suitable technique, algorithm or software program, for example, the randomForest algorithm. Without wishing to be bound by this theory, using the methods and compositions herein, the selected line-specific genes are expected to have higher confidence values associated with them, meaning line-specific genes identified through these processes are more likely to be validated and not generate false-positive or random line-specific gene candidates. In some examples, the selected line-specific genes are found in more than one type of tissue and are further compared to determine whether they are tissue agnostic line-specific genes. See, for example, Example 1.

In the present disclosure, the validation rate of obtaining a line-specific gene that confers upon a plant at least one agronomic characteristic by screening line-specific genes identified by the methods disclosed herein may be at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30%. In the present disclosure, the validation rate of obtaining a line-specific gene that confers upon a plant at least one first agronomic characteristic by screening line-specific genes identified by the methods disclosed herein may be at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30%. “Validation rate” as used herein refers to the rate of identifying genes showing desired phenotype in planta from the pool of candidate genes identified by any screening strategy. “Phenotype” means the detectable characteristics of a cell or organism.

For example, for a screening strategy that identifies putative candidate genes that may exhibit desired phenotype based on differential gene expression between stressed and non-stressed plants, the validation rate would be the number of genes that actually exhibit the desired phenotype in planta, compared to the total number of candidate genes identified that may show the desired phenotype identified on the basis of the differential expression experiment only.

For the purposes of the current disclosure, the validation rate refers to the number of line-specific or cluster-specific genes that show the desired phenotype in planta, compared to the total number of candidate line-specific genes or cluster-specific genes identified by the method disclosed herein.

The present disclosure includes a method of identifying at least one cluster-specific gene from a plurality of plants.

The term “cluster-specific gene” as used herein refers to a gene that shows perturbed expression in one first cluster of plants, but doesn't show the same perturbation of expression in at least one second cluster of plants, wherein a single plurality or group of plants comprises both the first and the at least one second cluster. The term “cluster-specific gene” is used interchangeably herein with the term “cluster-specific marker” (CSM) herein. The cluster-specific gene that shows perturbation of expression in the first cluster of plants, may not show the same perturbation of expression in at least a second, in at least a third, in at least a fourth, in at least a fifth, or at least an “nth” cluster. All these clusters used for identifying a cluster-specific gene and showing differential expression of the cluster-specific gene are in the same plurality or group of plants.

A plurality or group of plants that is used for identifying a cluster-specific gene comprises plants that show an alteration in at least one first agronomic trait or characteristic.

As used herein, the term “cluster” of plants means a group of plants, wherein the clustering of plants refers to organizing plants from a population of plants into groups, such that plants in the same group or cluster are more similar (in some sense or another) to each other than to those in other groups (clusters). For identifying a cluster specific gene, the plants from a plurality or population of plants are clustered or organized into groups.

Expression data for the line-specific genes for use in identifying cluster-specific genes may be collected or obtained from previously stored data. Data processing can be performed using any suitable techniques and in any number of steps, for example, filtering and normalizing, for example, as described for the primary gene expression data elsewhere herein.

Statistical processing and application of algorithms can be used to facilitate the data processing, analysis and comparison of the line-specific gene expression data.

The line-specific genes may be ranked, scored or otherwise assigned a value for example, an importance value, using any suitable technique, algorithm or software program, for example, the randomForest algorithm, and the higher ranking genes used for further analysis, for example, cluster analysis.

For the purposes of the current disclosure, the clustering of plants may be done on the basis of at least one criterion selected from the group consisting of the following three criteria:

1. All Plants in a Single Cluster Exhibit Similar Agronomic Characteristics, or Similar Alteration in Agronomic Characteristics, when Compared to a Control Plant:

The agronomic characteristics may be any agronomic characteristics, a few non-limiting examples of which are such as stress resistance, root architecture, shoot architecture, staygreen phenotype, ABA sensitivity and biomass.

Plants of one cluster can exhibit alteration in any number of agronomic characteristics, when compared to a control plant, wherein all plants of one cluster exhibit the same alteration in at least the same “n” number of agronomic characteristics. Plants of one cluster can exhibit alteration in at least one second, at least one third, at least one fourth agronomic characteristic.

Any assay that can be used for validating or testing any agronomic characteristic of a plant, can be used for clustering of plants. A non-limiting example of this would be, the plants for a population of plants that exhibit paraquat resistance and ABA-sensitivity may be clustered into a first cluster, and the plants that do not exhibit paraquat resistance and ABA-sensitivity may be clustered into a second cluster. Such assays are widely known and used for screening plant populations. Many of these assays have been described in literature. Examples of such assays include, but are not limited to osmotic stress assay, low nitrogen stress assay, root hydrotropism assay, ABA-sensitivity assay, root architecture assay, triple stress assay, paraquat resistance assay, soil root mass assay, soil drought assay, plant growth rate, plant biomass, seedling germination and growth under cold stress, thermotolerance assays (US Patent Publication No. US2014/0304854, WO 2010/020941, US2011/0035835, Roxas et al (1997) Acta Physiologiae Plantarum 19 (4):591-594, Larkindale et al Plant Physiology, June 2005, Vol. 138: 882-897),

A more comprehensive list of agronomic characteristics relevant to this disclosure are discussed elsewhere in this specification.

### 2. Similarity in Gene Expression Profiles:

In the present disclosure, the clustering of plants in a group or plurality of plants to identify a cluster-specific gene can be done on the basis of similarity of gene expression profiles between the plants. The similarity of gene expression profile is determined by the distance metric with a cluster bootstrap confidence value of at least 50%.

In the present disclosure, the similarity in gene expression used for clustering of plants may be determined by pattern-recognition algorithm. The pattern recognition algorithm may be a clustering algorithm.

Changes or perturbations in gene expression in a plant may be used to construct a clustering tree for purposes of grouping or clustering plants from a plurality of plants, with perturbation of specific primary genes, on the basis of similarities in gene expression. If the same set of genes is perturbed in the same direction in more than one plant, they are grouped into the same cluster. As used herein, the term “distance metric”, “distance matrix” and “dissimilarity matrix” are used interchangeably herein, and refer to the matrix that contains information about dissimilarity between two units.

“Distance matrix” may be defined as a matrix (two-dimensional array) containing the distances, taken pairwise, of a set of points. This matrix will have a size of N×N where N is the number of points, nodes or vertices (often in a graph).

As used herein the distance matrix is made by using the sample data and the gene data for each sample. A non-limiting example for this may be where the samples are the plants with perturbation of expression of different primary genes.

In the present disclosure, if distance between two units is below a given value, it may indicate a high similarity, whereas a distance equal to or greater than the given value may indicate low similarity.

All classifier and/or clustering algorithms use some distance or similarity measures to determine how close the samples or genes are to each other.

The distance metric can be determined by any machine learning algorithm. The distance metric may then be used by pattern recognition algorithms for grouping or clustering genes. In the present disclosure, the pattern recognition algorithm may be a clustering algorithm.

Examples of pattern-recognition algorithm that may be used for purposes of the current disclosure include, but are not limited to, connectivity based clustering, centroid based clustering and distribution based clustering. Some of the non-limiting examples of these clustering methods are hierarchical clustering (HC), UPGMA (“Unweighted Pair Group Method with Arithmetic Mean”, also known as average linkage clustering, Single-linkage clustering, Complete-linkage clustering (for connectivity based clustering), K-means (for Centroid based clustering), and Gaussian mixture models (for distribution based clustering, using the expectation-maximization algorithm)

All these different pattern recognition algorithms that may be used for the purposes of the current disclosure are well known in the art (US2010/0280987)

The genes being analyzed for the purposes of the method of the present disclosure may be grouped or re-ordered into co-varying sets. The genes and/or response profiles are each grouped by means of a pattern recognition procedure or algorithm, most preferably by means of a clustering procedure or algorithm. Such algorithms are well known to those of skill in the art, and are reviewed, e.g., by Becker, R. A., Chambers, J. M. and Wilks, A. R. (1988) The New S Language. Wadsworth & Brooks/Cole. (S version.), Everitt, B. (1974). Cluster Analysis. London: Heinemann Educ. Books, Hartigan, J. A. (1975). Clustering Algorithms. New York: Wiley, Sneath, P. H. A. and R. R. Sokal (1973). Numerical Taxonomy. San Francisco: Freeman, Anderberg, M. R. (1973). Cluster Analysis for Applications. Academic Press: New York, McQuitty, L. L. (1966) Educational and Psychological Measurement, 26, 825-831, US Patent publication No. US20030211475). Such algorithms include, for example, hierarchical agglomerative clustering algorithms, the “k-means” algorithm of Hartigan (supra), and model-based clustering algorithms such as hclust by MathSoft, Inc.

In the present disclosure, the clustering analysis for gene expression analysis may be done using a hierarchical clustering algorithm, it may be done by using the hclust algorithm. The clustering algorithms used in the present disclosure may operate on tables of data containing gene expression measurements.

The clustering algorithms used in the present disclosure for gene expression analysis analyze such arrays or matrices to determine dissimilarities between the individual genes or between individual response profiles. For example, the dissimilarity between two primary genes i and j may be expressed mathematically as the “distance” Dᵢⱼ. A variety of distance metrics which are known to those skilled in the art may be used in the clustering algorithms of the present disclosure. For example, the Euclidian distance may be determined to cluster the primary genes, which would lead to determination of plant clusters based on similarity in gene expression profiles.

As used herein “bootstrap confidence value” and “bootstrap confidence interval” are used interchangeably herein.

Bootstrapping method is well known method for making statistical inferences, and is a randomization technique, that reolies on experimental replication (Kerr and Churchill PNAS Jul. 31, (2001) 98(16):8961-8965; US Patent publication No. US20030003450;

A “bootstrap probability of >50%” would mean that at least in more than 50% of the cases or iterations, plants with the perturbations of the same primary genes from one plurality of plants should cluster together.

### 3. Perturbation of Expression of Members of the Same Gene Family:

Clustering of plants from a plurality or population of plants can be done by determining if the plants exhibit perturbation of expression of members of the same gene family. For example, plants that exhibit perturbation of expression of the members of the same gene family can be clustered together. The perturbation may be overexpression or downregulation. As another example, plants that exhibit overexpression of the members of the same gene family can be clustered into a single cluster. A gene family, for the purposes of this disclosure can be defined herein as a group of similar DNA or peptide sequences wherein the sequence similarity might span across the full length of complete sequences or the similarity might be restricted to discontinuous parts of the sequences (conserved domains and motifs). A gene family may also be defined as a group of similar DNA or peptide sequences which are related to each other by sequence similarity and can be traced back in evolution to a common ancestor. A gene family may also be defined as a group of DNA or peptide sequences which have similar characteristics including sequence similarity, structural similarity, functional similarity, part of a specific biological pathway or process or subcellular localisation.

In the present disclosure the at least one first agronomic characteristic may be resistance to biotic or abiotic stress. The at least one first agronomic characteristic may be resistance to biotic stress. In the present disclosure it may be resistance to abiotic stress. In the present disclosure the abiotic stress may be drought stress or low nitrogen stress.

As used herein, the term “pathway” is intended to mean a set of system of components involved in two or more sequential molecular interactions that result in the production of a product or activity. As used herein, a pathway is defined as a set of genes responding in a coordinated fashion irrespective of the underlying mechanism. A pathway can produce a variety of products or activities that can include, for example, intermolecular interactions, changes in expression of a nucleic acid or polypeptide, the formation or dissociation of a complex, between two or more molecules, accumulation or destruction of a metabolic product, activation or deactivation of an enzyme or binding activity.

In the present disclosure, inducing a particular pathway may lead to an alteration in an agronomic characteristic in a plant, or may confer upon the plant in which the pathway has been induced, a phenotype. In the present disclosure, perturbation of expression of a primary gene in a plant or plant cell may induce at least one biological pathway in the plant or plant cell.

The method of identifying at least one cluster specific gene from a plurality of plants includes analyzing gene expression in the plants from the at least one first cluster of plants and the at least one second cluster of plants.

In the current disclosure, the step for analyzing gene expression data in any of the methods for identifying at least one line-specific gene or for identifying at least one cluster-specific gene may be done in specific tissues. Said line specific gene or cluster-specific gene identified from the plurality of plants may show perturbation of expression in all the tissues analyzed for gene expression.

In the current disclosure, the plurality of plants may comprise of at least two plants. The plurality of plants may comprise at least 10 plants. In the present disclosure, all plants in the plurality of plants may exhibit alteration in at least one first agronomic characteristic, wherein said all plants in said plurality of plants exhibit alteration in the same at least one first agronomic characteristic. In the present disclosure, all plants in the plurality of plants may exhibit alteration in at least one first agronomic characteristic, wherein said all plants in said plurality of plants do not exhibit alteration in the same at least one first agronomic characteristic.

The gene expression data from the at least one first cluster of plants is compared to the gene expression data from the at least one second cluster of plants. Cluster-specific genes that are perturbed in at least 80% of the plants from the at least one first cluster of plants, and perturbed in not more than 20% of the plants from the at least one second cluster of plants are identified. In some examples, the expression of the cluster specific gene identified is perturbed in not more than 10% of the plants from the at least one second cluster of plants.

At least one of the steps of the method for identifying a cluster-specific gene from a plurality of plants may be done manually. At least one step of the method may be done computationally. At least one step of the method may done by using a machine learning algorithm.

The method of identifying a cluster-specific gene further may comprise the step of selecting a cluster-specific gene, wherein the cluster-specific gene confers upon a plant an alteration in the at least one first agronomic characteristic, wherein the plant shows a perturbation in expression of the cluster-specific gene when compared to a control plant. In some embodiments, the alteration in the at least one first agronomic characteristic in each plant in the plurality of plants may be due to perturbation of expression of a different gene. In some embodiments, the alteration in the at least one first agronomic characteristic in each plant in the plurality of plants may be due to perturbation of expression of the same gene.

In the present disclosure, the validation rate of obtaining a cluster-specific gene that confers upon a plant at least one agronomic characteristic by screening cluster-specific genes identified by the methods disclosed herein may be at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30%. In the present disclosure, the validation rate of obtaining a line-specific gene that confers upon a plant at least one first agronomic characteristic by screening cluster-specific genes identified by the methods disclosed herein may be at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30%.

Without wishing to be bound by this theory, using the methods and compositions herein, the selected cluster-specific genes are expected to have higher confidence values associated with them, and more likely to have validation rates and not generate false-positive or random cluster-specific gene candidates. Cluster-specific genes may be identified and selected and used for further analysis and testing.

As described herein, primary genes, line-specific genes, and/or cluster-specific genes including those existing or identified using the methods described here, may be used in any number of ways. In some examples, the primary genes, line-specific genes, and/or cluster-specific genes may be modified to create variants for further testing and evaluation of phenotype, such as agronomic characteristic, and effect on expression level and temporal and spatial expression. In some examples, modifications are made to orthologs or homologs of primary genes, line-specific genes, or cluster-specific genes.

Any suitable approach or technique may be used to introduce or create a polynucleotide encoding a transcript of a primary gene, a line-specific or a cluster-specific gene identified by any of the methods disclosed herein in a plant. For example, the polynucleotide may be introduced or created in the plant by modifying a regulatory element, a non-coding sequence or coding sequence or combinations thereof in an endogenous gene, a pre-existing recombinant sequence within the plant genome or introducing a recombinant sequence into the plant genome. In an embodiment, the polynucleotide is codon-optimized for expression, for example, to increase expression in a plant, for example, monocot or dicot codon-optimized. In an embodiment, the polynucleotide encoding a transcript of a line-specific or cluster-specific gene identified by any of the methods disclosed herein in a plant is a homolog or ortholog of a primary gene, a line-specific or cluster-specific gene identified by any of the methods disclosed herein. In an embodiment, the present disclosure includes a recombinant DNA construct comprising the polynucleotide, wherein the polynucleotide is operably linked to a heterologous regulatory element, and wherein said recombinant DNA construct confers upon a plant comprising said recombinant DNA construct at least one phenotype, wherein the phenotype is selected from the group consisting of: increased yield, increased productivity and increased stress resistance, when compared to a control plant. The present disclosure includes a plant comprising the recombinant DNA construct or polynucleotide encoding the transcript of a line-specific or cluster-specific gene, wherein the plant exhibits alteration in at least one phenotype, wherein the phenotype is selected from the group consisting of: increased yield, increased productivity and increased stress resistance, when compared to a control plant.

The current disclosure includes the use of the polynucleotide encoding the transcript of a line-specific or cluster-specific gene or the recombinant DNA construct disclosed herein, to produce a plant that exhibits alteration in at least one phenotype, wherein the phenotype is selected from the group consisting of: increased yield, increased productivity and increased stress resistance, when compared to a control plant.

Plants expressing these the line-specific genes, the cluster-specific genes, or variants thereof may be evaluated under various conditions, e.g. drought, low nitrogen, etc, in assays, greenhouse or field conditions. In another example, the line-specific genes, the cluster-specific genes, or variants thereof may be used as primary genes in the plants and methods described herein to facilitate the identification of additional line-specific genes or cluster-specific genes.

In some examples, the expression of the line-specific genes, the cluster-specific genes, or variants thereof in plants may be further perturbed using various techniques and approaches described herein and known to one in the art, for example, expressing the line-specific genes, the cluster-specific genes, or variants thereof using different promoters, e.g. of different strength and/or tissue-specificity, and evaluating the impact on the agronomic characteristic of the plant under various conditions.

Abiotic stress may be at least one condition selected from the group consisting of: drought, water deprivation, flood, high light intensity, high temperature, low temperature, salinity, etiolation, defoliation, heavy metal toxicity, anaerobiosis, nutrient deficiency, nutrient excess, UV irradiation, atmospheric pollution (e.g., ozone) and exposure to chemicals (e.g., paraquat) that induce production of reactive oxygen species (ROS).

Examples of other abiotic stress conditions include, but are not limited to, osmotic stress, paraquat stress, triple stress, low temperature stress and drought stress. In the present disclosure, the plants show at least one phenotype selected from the group consisting of increased tolerance to triple stress, altered root hydrotropism characteristics, increased percentage germination under cold conditions, increased paraquat tolerance, altered ABA response and increased tolerance to osmotic stress.

“Drought” refers to a decrease in water availability to a plant that, especially when prolonged, can cause damage to the plant or prevent its successful growth (e.g., limiting plant growth or seed yield).

The terms “drought”, “drought stress”, “low water availability”, “water stress” and “reduced water availability” are used interchangeably herein, and refer to less water availability to the plant than what is required for optimal growth and productivity.

“Drought tolerance” is a trait of a plant to survive under drought conditions over prolonged periods of time without exhibiting substantial physiological or physical deterioration.

“Drought tolerance activity” of a polypeptide indicates that over-expression of the polypeptide in a transgenic plant confers increased drought tolerance to the transgenic plant relative to a reference or control plant.

“Increased drought tolerance” of a plant is measured relative to a reference or control plant, and is a trait of the plant to survive under drought conditions over prolonged periods of time, without exhibiting the same degree of physiological or physical deterioration relative to the reference or control plant grown under similar drought conditions. Typically, when a transgenic plant comprising a recombinant DNA construct or suppression DNA construct in its genome exhibits increased drought tolerance relative to a reference or control plant, the reference or control plant does not comprise in its genome the recombinant DNA construct or suppression DNA construct.

“Triple stress” as used herein refers to the abiotic stress exerted on the plant by the combination of drought stress, high temperature stress and high light stress.

The terms “heat stress” and “high temperature stress” are used interchangeably herein, and are defined as where ambient temperatures are hot enough for sufficient time that they cause damage to plant function or development, which might be reversible or irreversible in damage. “High temperature” can be either “high air temperature” or “high soil temperature”, “high day temperature” or “high night temperature, or a combination of more than one of these.

In the present disclosure, the ambient temperature may be in the range of 30° C. to 36° C. In the present disclosure, the duration for the high temperature stress may be in the range of 1-16 hours.

“High light intensity” and “high irradiance” and “light stress” are used interchangeably herein, and refer to the stress exerted by subjecting plants to light intensities that are high enough for sufficient time that they cause photoinhibition damage to the plant.

In the present disclosure, the light intensity may be in the range of 250 μE to 450 μE. In the present disclosure, the duration for the high light intensity stress may be in the range of 12-16 hours.

“Triple stress tolerance” is a trait of a plant to survive under the combined stress conditions of drought, high temperature and high light intensity over prolonged periods of time without exhibiting substantial physiological or physical deterioration.

“Nitrogen stress tolerance” is a trait of a plant and refers to the ability of the plant to survive under nitrogen limiting conditions.

“Increased nitrogen stress tolerance” of a plant is measured relative to a reference or control plant, and means that the nitrogen stress tolerance of the plant is increased by any amount or measure when compared to the nitrogen stress tolerance of the reference or control plant.

A “nitrogen stress tolerant plant” is a plant that exhibits nitrogen stress tolerance. A nitrogen stress tolerant plant may be a plant that exhibits an increase in at least one agronomic characteristic relative to a control plant under nitrogen limiting conditions.

“Increased stress tolerance” of a plant is measured relative to a reference or control plant, and is a trait of the plant to survive under stress conditions over prolonged periods of time, without exhibiting the same degree of physiological or physical deterioration relative to the reference or control plant grown under similar stress conditions.

A plant with “increased stress tolerance” can exhibit increased tolerance to one or more different stress conditions.

“Stress tolerance activity” of a polypeptide indicates that over-expression of the polypeptide in a transgenic plant confers increased stress tolerance to the transgenic plant relative to a reference or control plant.

A polypeptide with a certain activity, such as a polypeptide with one or more than one activity selected from the group consisting of: increased triple stress tolerance, increased drought stress tolerance, increased nitrogen stress tolerance, increased osmotic stress tolerance, altered ABA response, altered root architecture, increased tiller number; indicates that overexpression of the polypeptide in a plant confers the corresponding phenotype to the plant relative to a reference or control plant. For example, a plant overexpressing a polypeptide with “altered ABA response activity”, would exhibit the phenotype of “altered ABA response”, when compared to a control or reference plant.

The term “plant productivity” as used herein is defined as the dry weight per unit of ground area), or the yield per unit of ground area. Thus, for purposes of the present disclosure, improved or increased plant productivity may refer to improvements in biomass or yield of leaves, stems, grain, fruit, vegetables, flowers, or other plant parts harvested or used for various purposes, and improvements in growth of plant parts, including stems, leaves and roots. For example, when referring to food crops, such as grains, fruits or vegetables, plant productivity may refer to the yield of grain, fruit, vegetables or seeds harvested from a particular crop. For crops such as pasture, plant productivity may refer to growth rate, plant density or the extent of groundcover.

“Plant growth” refers to the growth of any, plant part, including stems, leaves and roots. Growth may refer to the rate of growth of any one of these plant parts (Zelitch, I. Proc. Nat. Acad. Sci. USA Vol. 70, No. 2, pp. 579-584, February 1973). Regulating the activity of genes that can affect plant architecture, development or yield could likely be the key to increasing plant productivity

Increased biomass can be measured, for example, as an increase in plant height, plant total leaf area, plant fresh weight, plant dry weight or plant seed yield, as compared with control plants.

The ability to increase the biomass or size of a plant would have several important commercial applications. Crop species may be generated that produce larger cultivars, generating higher yield in, for example, plants in which the vegetative portion of the plant is useful as food, biofuel or both.

Increased leaf size may be of particular interest. Increasing leaf biomass can be used to increase production of plant-derived pharmaceutical or industrial products. An increase in total plant photosynthesis is typically achieved by increasing leaf area of the plant. Additional photosynthetic capacity may be used to increase the yield derived from particular plant tissue, including the leaves, roots, fruits or seed, or permit the growth of a plant under decreased light intensity or under high light intensity.

Modification of the biomass of another tissue, such as root tissue, may be useful to improve a plant's ability to grow under harsh environmental conditions, including drought or nutrient deprivation, because larger roots may better reach water or nutrients or take up water or nutrients.

For some ornamental plants, the ability to provide larger varieties would be highly desirable. For many plants, including fruit-bearing trees, trees that are used for lumber production, or trees and shrubs that serve as view or wind screens, increased stature provides improved benefits in the forms of greater yield or improved screening.

The growth and emergence of maize silks has a considerable importance in the determination of yield under drought (Fuad-Hassan et al. 2008 Plant Cell Environ. 31:1349-1360). When soil water deficit occurs before flowering, silk emergence out of the husks is delayed while anthesis is largely unaffected, resulting in an increased anthesis-silking interval (ASI) (Edmeades et al. 2000 Physiology and Modeling Kernel set in Maize (eds M. E. Westgate & K. Boote; CSSA (Crop Science Society of America) Special Publication No.29. Madison, Wis.: CSSA, 43-73). Selection for reduced ASI has been used successfully to increase drought tolerance of maize (Edmeades et al. 1993 Crop Science 33: 1029-1035; Bolanos & Edmeades 1996 Field Crops Research 48:65-80; Bruce et al. 2002 J. Exp. Botany 53:13-25).

Terms used herein to describe thermal time include “growing degree days” (GDD), “growing degree units” (GDU) and “heat units” (HU).

In the present disclosure, “yield” may be measured in many ways, including, for example, test weight, seed weight, seed number per plant, seed number per unit area (i.e. seeds, or weight of seeds, per acre), bushels per acre, tonnes per acre, tons per acre, kilo per hectare.

In the present disclosure, the plant with perturbation of expression of at least one line-specific gene and/or at least one cluster-specific gene may exhibit less yield loss relative to the control plants, for example, at least 25%, at least 20%, at least 15%, at least 10% or at least 5% less yield loss, under water limiting conditions, or would have increased yield, for example, at least 5%, at least 10%, at least 15%, at least 20% or at least 25% increased yield, relative to the control plants under water non-limiting conditions.

In the present disclosure, the plant may exhibit less yield loss relative to the control plants, for example, at least 25%, at least 20%, at least 15%, at least 10% or at least 5% less yield loss, under stress conditions, or would have increased yield, for example, at least 5%, at least 10%, at least 15%, at least 20% or at least 25% increased yield, relative to the control plants under non-stress conditions. The stress may be selected from the group consisting of drought stress, triple stress, nitrogen stress and osmotic stress.

One of ordinary skill in the art is familiar with protocols for simulating stress conditions and for evaluating stress tolerance of plants that have been subjected to simulated or naturally-occurring stress conditions. For example, one can simulate drought stress conditions by giving plants less water than normally required or no water over a period of time, and one can evaluate drought tolerance by looking for differences in physiological and/or physical condition, including (but not limited to) vigor, growth, size, or root length, or in particular, leaf color or leaf area size. Other techniques for evaluating drought tolerance include measuring chlorophyll fluorescence, photosynthetic rates and gas exchange rates. In any of the methods of the present disclosure, the step of selecting an alteration of an agronomic characteristic in a progeny plant, if applicable, may comprise selecting a progeny plant that exhibits an alteration of at least one agronomic characteristic when compared, under varying environmental conditions, to a control plant not comprising the polynucleotide encoding the primary gene, line-specific gene, or cluster-specific gene or recombinant DNA construct or a control plant not perturbed in the polynucleotide encoding the primary gene, line-specific gene, or cluster-specific gene or a control plant not having an alteration in the at the least one agronomic characteristic.

A drought stress experiment may involve a chronic stress (i.e., slow dry down) and/or may involve two acute stresses (i.e., abrupt removal of water) separated by a day or two of recovery. Chronic stress may last 8-10 days. Acute stress may last 3-5 days. The following variables may be measured during drought stress and well watered treatments of transgenic plants and relevant control plants:

The Examples below describe some representative protocols and techniques for simulating drought conditions and/or evaluating drought tolerance.

One can also evaluate drought tolerance by the ability of a plant to maintain sufficient yield (at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% yield) in field testing under simulated or naturally-occurring drought conditions (e.g., by measuring for substantially equivalent yield under drought conditions compared to non-drought conditions, or by measuring for less yield loss under drought conditions compared to a control or reference plant).

One of ordinary skill in the art would readily recognize a suitable control or reference plant to be utilized when assessing or measuring an agronomic characteristic or phenotype of a plant of the present disclosure in which a control plant is utilized (e.g., compositions or methods as described herein). For example, by way of non-limiting illustrations:

The commercial development of genetically improved germplasm has also advanced to the stage of introducing multiple traits into crop plants, often referred to as a gene stacking approach. In this approach, multiple genes conferring different characteristics of interest can be introduced into a plant. Gene stacking can be accomplished by many means including but not limited to co-transformation, retransformation, and crossing lines with different transgenes.

In hybrid seed propagated crops, mature transgenic plants can be self-pollinated to produce a homozygous inbred plant. The inbred plant produces seed containing the newly introduced polynucleotide encoding a transcript of a line-specific or cluster-specific gene identified by any of the methods disclosed herein or a recombinant DNA construct (or suppression DNA construct). These seeds can be grown to produce plants that would exhibit an altered agronomic characteristic (e.g., an increased agronomic characteristic optionally under stress conditions), or used in a breeding program to produce hybrid seed, which can be grown to produce plants that would exhibit such an altered agronomic characteristic. The seeds may be maize seeds. The stress condition may be selected from the group of drought stress, triple stress and osmotic stress. The plant may be a monocotyledonous or dicotyledonous plant, for example, a maize or soybean plant. The plant may also be sunflower, sorghum, canola, wheat, alfalfa, cotton, rice, barley, millet, sugar cane or switchgrass.

In some examples, the methods described herein include growing a plant that exhibits perturbation of expression of either a primary gene, and/or a line-specific gene, and/or a cluster-specific gene for further testing and evaluation of the agronomic characteristic. In some instances, the method includes using the selected plant that exhibits perturbation of expression of either a primary gene, and/or a line-specific gene, and/or a cluster-specific gene in a plant breeding program. For example, the plant may be used in recurrent selection, bulk selection, mass selection, backcrossing, pedigree breeding, open pollination breeding, restriction fragment length polymorphism enhanced selection, genetic marker enhanced selection, double haploids and transformation. In some instances the plant may be crossed with another plant or back-crossed so that the gene can be introgressed into the plant by sexual outcrossing or other conventional breeding methods.

In some instances, the primary gene, and/or a line-specific gene, and/or a cluster-specific gene may be used as a marker for use in marker-assisted selection in a breeding program to produce plants that exhibit an alteration of at least one agronomic characteristic or exhibit perturbation of expression of a primary gene, and/or a line-specific gene, and/or a cluster-specific gene. The perturbation of expression in the primary gene, line-specific or cluster-specific gene may be used as marker for the first plant to distinguish the first plant from the rest of the plants in the plurality of plants.

In any of the methods of the present disclosure, the step of selecting an alteration of an agronomic characteristic in a plant that exhibits perturbation of expression of either a primary gene, and/or a line-specific gene, and/or a cluster-specific gene, if applicable, may comprise selecting a plant that exhibits an alteration of at least one agronomic characteristic when compared, under varying environmental conditions, to a control plant not exhibiting perturbation of expression of a primary gene, and/or a line-specific gene, and/or a cluster-specific gene.

A method of producing seed (for example, seed that can be sold as a drought tolerant product offering) comprising any of the preceding methods, and further comprising obtaining seeds from said progeny plant, wherein said seeds comprise in their genome said polynucleotide encoding a transcript from the line-specific gene, and/or a cluster-specific gene or a recombinant DNA construct (or suppression DNA construct).

A method of producing oil or a seed by-product, or both, from a seed, the method comprising extracting oil or a seed by-product, or both, from a seed that comprises a said polynucleotide encoding a transcript from the line-specific gene, and/or a cluster-specific gene or a recombinant DNA construct, wherein the recombinant DNA construct comprises a polynucleotide encoding a transcript of a line-specific or cluster-specific gene identified by any of the methods disclosed herein, wherein the polynucleotide is operably linked to at least one heterologous regulatory element. The seed may be obtained from a plant that comprises the polynucleotide encoding a transcript from the line-specific gene, and/or a cluster-specific gene or a recombinant DNA construct, wherein the plant exhibits at least one phenotype selected from the group consisting of increased yield, increased productivity and increased stress resistance, when compared to a control plant not comprising the recombinant DNA construct. The polypeptide may exhibit perturbation of expression in at least one tissue of the plant, or during at least one condition of abiotic or biotic stress, or both. The plant may be selected from the group consisting of: maize, soybean, sunflower, sorghum, canola, wheat, alfalfa, cotton, rice, barley, millet, sugar cane and switchgrass. The oil or the seed by-product, or both, may comprise the polynucleotide encoding a transcript from the line-specific gene, and/or a cluster-specific gene or the recombinant DNA construct. The plant may be a monocotyledonous or dicotyledonous plant, for example, a maize or soybean plant. The plant may also be sunflower, sorghum, canola, wheat, alfalfa, cotton, rice, barley, millet, sugar cane or sorghum. The seed may be a maize or soybean seed, for example, a maize hybrid seed or maize inbred seed.

Also provided is a method of selecting for (or identifying) an alteration of an agronomic characteristic in a plant, where the method comprises (a) obtaining a transgenic plant comprising in its genome a polynucleotide encoding a transcript of a line-specific or cluster-specific gene identified by any of the methods disclosed herein or a recombinant DNA construct comprising a polynucleotide operably linked to at least one heterologous regulatory element, wherein said polynucleotide encodes a transcript of a line-specific or cluster-specific gene identified by any of the methods disclosed herein; (b) obtaining a progeny plant derived from said transgenic plant, wherein the progeny plant comprises in its genome the polynucleotide or recombinant DNA construct; and (c) selecting (or identifying) the progeny plant that exhibits an alteration in the at least one first agronomic characteristic when compared, under stress or non-stress conditions, wherein the stress is selected from the group consisting of abiotic stress or biotic stress, to a control plant not comprising the polynucleotide or recombinant DNA construct. The agronomic characteristic may be the at least one first agronomic characteristic or the at least one second agronomic characteristic for purposes of the methods disclosed herein. In any of the methods of the present disclosure, the at least one agronomic characteristic may be selected from the group comprising or consisting of: abiotic stress tolerance, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, leaf number, tiller number, growth rate, first pollen shed time, first silk emergence time, anthesis silking interval (ASI), stalk diameter, root architecture, staygreen, relative water content, water use, water use efficiency, dry weight of either main plant, tillers, primary ear, main plant and tillers or cobs; rows of kernels, total plant weight, kernel weight, kernel number, salt tolerance, chlorophyll content, flavonol content, number of yellow leaves, early seedling vigor and seedling emergence under low temperature stress. The alteration of at least one agronomic characteristic may be an increase in yield, greenness or biomass. These agronomic characteristics maybe measured at any stage of the plant development. One or more of these agronomic characteristics may be measured under stress or non-stress conditions, and may show alteration on overexpression of the polynucleotides or recombinant constructs disclosed herein.

A composition of the present disclosure includes a transgenic microorganism, cell, plant, and seed comprising the polynucleotide encoding a transcript of a line-specific or cluster-specific gene identified by any of the methods disclosed herein or the recombinant DNA construct. The cell may be eukaryotic, e.g., a yeast, insect or plant cell, or prokaryotic, e.g., a bacterial cell. A composition of the present disclosure is a plant made by any of the methods disclosed herein.

Accordingly, a composition of the present disclosure is a plant comprising in its genome any of the polynucleotides encoding a transcript of a line-specific or cluster-specific gene identified by any of the methods disclosed herein or recombinant DNA constructs (including any of the suppression DNA constructs) of the present disclosure (such as any of the constructs discussed above or below).

Compositions also include any progeny of the plant, and any seed obtained from the plant or its progeny, wherein the progeny or seed comprises within its genome the polynucleotide encoding a transcript of a line-specific or cluster-specific gene identified by any of the methods disclosed herein or the recombinant DNA construct (or suppression DNA construct). Progeny includes subsequent generations obtained by self-pollination or out-crossing of a plant. Progeny also includes hybrids and inbreds.

As used herein the terms non-genomic nucleic acid sequence or non-genomic nucleic acid molecule generally refer to a nucleic acid molecule that has one or more change in the nucleic acid sequence compared to a native or genomic nucleic acid sequence. In the present disclosure, the change to a native or genomic nucleic acid molecule may include but is not limited to: changes in the nucleic acid sequence due to the degeneracy of the genetic code; codon optimization of the nucleic acid sequence for expression in plants; changes in the nucleic acid sequence to introduce at least one amino acid substitution, insertion, deletion and/or addition compared to the native or genomic sequence; removal of one or more intron associated with a genomic nucleic acid sequence; insertion of one or more heterologous introns; deletion of one or more upstream or downstream regulatory regions associated with a genomic nucleic acid sequence; insertion of one or more heterologous upstream or downstream regulatory regions; deletion of the 5′ and/or 3′ untranslated region associated with a genomic nucleic acid sequence; and insertion of a heterologous 5′ and/or 3′ untranslated region.

As used herein, the term “gene” has its meaning as understood in the art. The term “gene” may include gene regulatory sequences (examples of regulatory sequences include but are not limited to promoter, enhancers, introns etc.), and may refer to genomic sequences, RNA or cDNA. For the purposes of the current disclosure the term “gene” encompasses nucleic acids that can code for a polypeptide (mRNA), as well as non-polypeptide coding RNAs. Examples of non-coding RNAs encoded by the genes relevant to the current disclosure include, but are not limited to, transfer RNA (tRNA), rRNA, microRNA (miRNA), long non-coding RNA (lincRNAs) or any other kind of RNA (WO2008121866, US2014/0315985).

“Allele” is one of several alternative forms of a gene occupying a given locus on a chromosome. When the alleles present at a given locus on a pair of homologous chromosomes in a diploid plant are the same that plant is homozygous at that locus. If the alleles present at a given locus on a pair of homologous chromosomes in a diploid plant differ that plant is heterozygous at that locus. If a transgene is present on one of a pair of homologous chromosomes in a diploid plant that plant is hemizygous at that locus.

Allelic variants encompass Single nucleotide polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms.

“cDNA” generally refers to a DNA that is complementary to and synthesized from a mRNA template using the enzyme reverse transcriptase. The cDNA can be single-stranded or converted into the double-stranded form using the Klenow fragment of DNA polymerase I.

“Coding region” generally refers to the portion of a messenger RNA (or the corresponding portion of another nucleic acid molecule such as a DNA molecule) which encodes a protein or polypeptide. “Non-coding region” generally refers to all portions of a messenger RNA or other nucleic acid molecule that are not a coding region, including but not limited to, for example, the promoter region, 5′ untranslated region (“UTR”), 3′ UTR, intron and terminator. The terms “coding region” and “coding sequence” are used interchangeably herein. The terms “non-coding region” and “non-coding sequence” are used interchangeably herein.

The terms “dicot” and “dicotyledonous plant” are used interchangeably herein. A dicot of the current disclosure includes the following families: Brassicaceae, Leguminosae, and Solanaceae.

The terms “entry clone” and “entry vector” are used interchangeably herein.

An “Expressed Sequence Tag” (“EST”) is a DNA sequence derived from a cDNA library and therefore is a sequence which has been transcribed. An EST is typically obtained by a single sequencing pass of a cDNA insert. The sequence of an entire cDNA insert is termed the “Full-Insert Sequence” (“FIS”). A “Contig” sequence is a sequence assembled from two or more sequences that can be selected from, but not limited to, the group consisting of an EST, FIS and PCR sequence. A sequence encoding an entire or functional protein is termed a “Complete Gene Sequence” (“CGS”) and can be derived from an FIS or a contig.

“Expression” generally refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

The terms “full complement” and “full-length complement” are used interchangeably herein, and refer to a complement of a given nucleotide sequence, wherein the complement and the nucleotide sequence consist of the same number of nucleotides and are 100% complementary.

As used herein, the term “gene” has its meaning as understood in the art. The term “gene” may include gene regulatory sequences (examples of regulatory sequences include but are not limited to promoter, enhancers, introns etc), and may refer to genomic sequences, RNA or cDNA. For the purposes of the current disclosure the term “gene” encompasses nucleic acids that can code for a polypeptide (mRNA), as well as non-polypeptide coding RNAs. Examples of non-coding RNAs encoded by the genes relevant to the current disclosure include, but are not limited to, transfer RNA (tRNA), rRNA, microRNA (miRNA), long non-coding

“Genome” as it applies to plant cells encompasses not only chromosomal DNA found within the nucleus, but organelle DNA found within subcellular components (e.g., mitochondrial, plastid) of the cell.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

“Isolated” generally refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

“Messenger RNA (mRNA)” generally refers to the RNA that is without introns and that can be translated into protein by the cell.

“Mature” protein generally refers to a post-translationally processed polypeptide; i.e., one from which any pre- or pro-peptides present in the primary translation product have been removed.

The terms “monocot” and “monocotyledonous plant” are used interchangeably herein. A monocot of the current disclosure includes the Gramineae.

“Plant” includes reference to whole plants, plant organs, plant tissues, plant propagules, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

“Polynucleotide”, “nucleic acid sequence”, “nucleotide sequence”, or “nucleic acid fragment” are used interchangeably and is a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

“Operably linked” generally refers to the association of nucleic acid fragments in a single fragment so that the function of one is regulated by the other. For example, a promoter is operably linked with a nucleic acid fragment when it is capable of regulating the transcription of that nucleic acid fragment.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, sulfation, gamma-carboxylation of glutamic acid residues, hydroxylation and ADP-ribosylation.

“Precursor” protein generally refers to the primary product of translation of mRNA; i.e., with pre- and pro-peptides still present. Pre- and pro-peptides may be and are not limited to intracellular localization signals.

“Propagule” includes all products of meiosis and mitosis able to propagate a new plant, including but not limited to, seeds, spores and parts of a plant that serve as a means of vegetative reproduction, such as corms, tubers, offsets, or runners. Propagule also includes grafts where one portion of a plant is grafted to another portion of a different plant (even one of a different species) to create a living organism. Propagule also includes all plants and seeds produced by cloning or by bringing together meiotic products, or allowing meiotic products to come together to form an embryo or fertilized egg (naturally or with human intervention).

“Progeny” comprises any subsequent generation of a plant. “Recombinant” generally refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention. “Promoter” generally refers to a nucleic acid fragment capable of controlling transcription of another nucleic acid fragment.

“Promoter functional in a plant” is a promoter capable of controlling transcription in plant cells whether or not its origin is from a plant cell.

“Tissue-specific promoter” and “tissue-preferred promoter” are used interchangeably, and refer to a promoter that is expressed predominantly but not necessarily exclusively in one tissue or organ, but that may also be expressed in one specific cell.

“Developmentally regulated promoter” generally refers to a promoter whose activity is determined by developmental events.

“Recombinant DNA construct” generally refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature. The terms “recombinant DNA construct” and “recombinant construct” are used interchangeably herein.

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences. The terms “regulatory sequence” and “regulatory element” are used interchangeably herein.

A “trait” generally refers to a physiological, morphological, biochemical, or physical characteristic of a plant or a particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, or by agricultural observations such as osmotic stress tolerance or yield.

A “transformed cell” is any cell into which a nucleic acid fragment (e.g., a recombinant DNA construct) has been introduced.

“Transformation” as used herein generally refers to both stable transformation and transient transformation.

“Stable transformation” generally refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” generally refers to the introduction of a nucleic acid fragment into the nucleus, or DNA-containing organelle, of a host organism resulting in gene expression without genetically stable inheritance.

“Transgenic” generally refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

“Transgenic plant” includes reference to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a recombinant DNA construct.

“Transgenic plant” also includes reference to plants which comprise more than one heterologous polynucleotide within their genome. Each heterologous polynucleotide may confer a different trait to the transgenic plant.

As mentioned elsewhere herein, the present disclosure encompasses the line-specific genes and cluster-specific genes identified by any of the methods disclosed herein. The primary genes, line-specific genes, and cluster-specific genes if desired, can isolated and analyzed using techniques known in the art, including sequence analysis, electrophoretic analysis, expression assays, and modified.

The current disclosure also encompasses the polynucleotides encoding the transcripts of the line-specific and/or cluster-specific genes, and the polypeptides encoded by the aforementioned genes and their transcripts. Also included in the current disclosure is polynucleotide encoding a transcript of a line-specific or cluster-specific gene identified by any of the methods disclosed herein, wherein said polynucleotide, upon perturbation of expression in a plant, confers upon said plant at least one phenotype, wherein the phenotype is selected from the group consisting of: increased yield, increased productivity and increased stress resistance, when compared to a control plant.

It is understood, as those skilled in the art will appreciate, that the disclosure encompasses more than the specific and exact sequences identified by the methods disclosed herein, for example, variants of these sequences in its regulatory, coding or non-coding sequences.

Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

Proteins derived by amino acid deletion, substitution, insertion and/or addition can be prepared when DNAs encoding their wild-type proteins are subjected to, for example, well-known site-directed mutagenesis (see, e.g., Nucleic Acid Research, Vol. 10, No. 20, p. 6487-6500, 1982, which is hereby incorporated by reference in its entirety). As used herein, the term “one or more amino acids” is intended to mean a possible number of amino acids which may be deleted, substituted, inserted and/or added by site-directed mutagenesis.

Site-directed mutagenesis may be accomplished, for example, as follows using a synthetic oligonucleotide primer that is complementary to single-stranded phage DNA to be mutated, except for having a specific mismatch (i.e., a desired mutation). Namely, the above synthetic oligonucleotide is used as a primer to cause synthesis of a complementary strand by phages, and the resulting duplex DNA is then used to transform host cells. The transformed bacterial culture is plated on agar, whereby plaques are allowed to form from phage-containing single cells. As a result, in theory, 50% of new colonies contain phages with the mutation as a single strand, while the remaining 50% have the original sequence. At a temperature which allows hybridization with DNA completely identical to one having the above desired mutation, but not with DNA having the original strand, the resulting plaques are allowed to hybridize with a synthetic probe labeled by kinase treatment. Subsequently, plaques hybridized with the probe are picked up and cultured for collection of their DNA.

Techniques for allowing deletion, substitution, insertion and/or addition of one or more amino acids in the amino acid sequences of biologically active peptides such as enzymes while retaining their activity include site-directed mutagenesis, as well as other techniques such as those for treating a gene with a mutagen, and those in which a gene is selectively cleaved to remove, substitute, insert or add a selected nucleotide or nucleotides, and then ligated or through genome editing approaches described herein and those available to one of ordinary skill in the art.

In another embodiment, compositions and methods include introducing a polynucleotide encoding the transcript of line-specific and/or cluster-specific gene into the plant genome, whereby the transcript is expressed from the polynucleotide. In some cases, the transcript produces a polypeptide. The polynucleotide can, but need not, be provided in a construct, e.g., a recombinant DNA construct, or suppression DNA construct, or can be introduced by other suitable techniques or approaches. The polynucleotide encoding the transcript of line-specific and/or cluster-specific gene may confer upon the plant at least one phenotype, wherein the phenotype is selected from the group consisting of: increased yield, increased productivity and increased stress resistance, when compared to a control plant. In some aspects, the present disclosure includes recombinant DNA constructs (including suppression DNA constructs) comprising the polynucleotides encoding the transcript of line-specific and/or cluster-specific gene. The transcript may be operably linked to at least one heterologous regulatory element. The recombinant construct may confer upon the plant at least one phenotype, wherein the phenotype is selected from the group consisting of: increased yield, increased productivity and increased stress resistance, when compared to a control plant.

The at least one heterologous regulatory element may comprise an enhancer sequence or a multimer of identical or different enhancer sequences. The at least one heterologous regulatory element may comprise one, two, three or four copies of the CaMV 35S enhancer. Suppression DNA constructs and silencing are described elsewhere herein and known to one skilled in the art.

The polynucleotide encoding the transcript of the line-specific gene and/or cluster-specific gene and the polypeptide encoded by the transcript may be from any plant species, for example, Arabidopsis thaliana, Zea mays, Glycine max, Glycine tabacina, Glycine soja, Glycine tomentella, Oryza sativa, Brassica napus, Sorghum bicolor, Saccharum officinarum, Triticum aestivum. These plant species are just exemplary, and not limiting examples of the plant species that can be used for the methods disclosed herein.

Regulatory Sequences:

A polynucleotide encoding a transcript of a line-specific or cluster-specific gene identified by any of the methods disclosed herein or recombinant DNA construct (including a suppression DNA construct) of the present disclosure may be further modified to affect its expression level, spatial or temporal pattern, for example, by modifying or introducing a regulatory element. Examples of various promoters and elements are described herein and known in the art.

In some aspects, the polynucleotide encoding a transcript of a line-specific or cluster-specific gene identified by any of the methods disclosed herein or recombinant DNA construct (including a suppression DNA construct) of the present disclosure comprise at least one regulatory sequence. In some examples, the regulatory sequence is heterologous with respect to the polynucleotide encoding a transcript of a line-specific or cluster-specific gene identified by any of the methods disclosed herein or recombinant DNA construct. In some examples, the regulatory sequence is heterologous with respect to the polynucleotide encoding a transcript of a line-specific or cluster-specific gene identified by any of the methods disclosed herein or recombinant DNA construct. A regulatory sequence may be a promoter.

Accordingly, in an embodiment, a plant comprises a modified regulatory element, coding sequence or non-coding sequence of the endogenous genes, of pre-existing recombinant sequences in the plant genome or of recombinant DNA constructs engineered to perturb the expression of one or more primary genes, line-specific genes, cluster-specific genes, including those line-specific genes or cluster-specific genes identified by the methods disclosed herein.

A number of promoters can be used with the polynucleotide encoding a transcript of a line-specific or cluster-specific gene identified by any of the methods disclosed herein or in recombinant DNA constructs of the present disclosure. The promoters can be selected based on the desired outcome, and may include constitutive, tissue-specific, inducible, or other promoters for expression in the host organism.

Promoters that cause a gene to be expressed in most cell types at most times are commonly referred to as “constitutive promoters”.

High level, constitutive expression of the candidate gene under control of the 35S or UBI promoter may have pleiotropic effects, although candidate gene efficacy may be estimated when driven by a constitutive promoter. Use of tissue-specific and/or stress-specific promoters may eliminate undesirable effects but retain the ability to enhance stress tolerance. This effect has been observed in Arabidopsis (Kasuga et al. (1999) Nature Biotechnol. 17:287-91).

Suitable constitutive promoters for use in a plant host cell include, for example, the core promoter of the Rsyn7 promoter and other constitutive promoters disclosed in WO 99/43838 and U.S. Pat. No. 6,072,050; the core CaMV 35S promoter (Odell et al., Nature 313:810-812 (1985)); rice actin (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)); ALS promoter (U.S. Pat. No. 5,659,026), the constitutive synthetic core promoter SCP1 (International Publication No. 03/033651) and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142; and 6,177,611.

In choosing a promoter to use in the methods of the disclosure, it may be desirable to use a tissue-specific or developmentally regulated promoter.

A tissue-specific or developmentally regulated promoter is a DNA sequence which regulates the expression of a DNA sequence selectively in the cells/tissues of a plant critical to tassel development, seed set, or both, and limits the expression of such a DNA sequence to the period of tassel development or seed maturation in the plant. Any identifiable promoter may be used in the methods of the present disclosure which causes the desired temporal and spatial expression.

Promoters which are seed or embryo-specific and may be useful include soybean Kunitz trypsin inhibitor (Kti3, Jofuku and Goldberg, Plant Cell 1:1079-1093 (1989)), patatin (potato tubers) (Rocha-Sosa, M., et al. (1989) EMBO J. 8:23-29), convicilin, vicilin, and legumin (pea cotyledons) (Rerie, W. G., et al. (1991) Mol. Gen. Genet. 259:149-157; Newbigin, E. J., et al. (1990) Planta 180:461-470; Higgins, T. J. V., et al. (1988) Plant. Mol. Biol. 11:683-695), zein (maize endosperm) (Schemthaner, J. P., et al. (1988) EMBO J. 7:1249-1255), phaseolin (bean cotyledon) (Segupta-Gopalan, C., et al. (1985) Proc. Natl. Acad. Sci. U.S.A. 82:3320-3324), phytohemagglutinin (bean cotyledon) (Voelker, T. et al. (1987) EMBO J. 6:3571-3577), B-conglycinin and glycinin (soybean cotyledon) (Chen, Z-L, et al. (1988) EMBO J. 7:297-302), glutelin (rice endosperm), hordein (barley endosperm) (Marris, C., et al. (1988) Plant Mol. Biol. 10:359-366), glutenin and gliadin (wheat endosperm) (Colot, V., et al. (1987) EMBO J. 6:3559-3564), and sporamin (sweet potato tuberous root) (Hattori, T., et al. (1990) Plant Mol. Biol. 14:595-604). Promoters of seed-specific genes operably linked to heterologous coding regions in chimeric gene constructions maintain their temporal and spatial expression pattern in transgenic plants. Such examples include Arabidopsis thaliana 2S seed storage protein gene promoter to express enkephalin peptides in Arabidopsis and Brassica napus seeds (Vanderkerckhove et al., Bio/Technology 7:L929-932 (1989)), bean lectin and bean beta-phaseolin promoters to express luciferase (Riggs et al., Plant Sci. 63:47-57 (1989)), and wheat glutenin promoters to express chloramphenicol acetyl transferase (Colot et al., EMBO J 6:3559-3564 (1987)). Endosperm preferred promoters include those described in e.g., U.S. Pat. No. 8,466,342; U.S. Pat. No. 7,897,841; and U.S. Pat. No. 7,847,160.

Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners.

Promoters for use include the following: 1) the stress-inducible RD29A promoter (Kasuga et al. (1999) Nature Biotechnol. 17:287-91); 2) the barley promoter, B22E; expression of B22E is specific to the pedicel in developing maize kernels (“Primary Structure of a Novel Barley Gene Differentially Expressed in Immature Aleurone Layers”. Klemsdal, S. S. et al., Mol. Gen. Genet. 228(1/2):9-16 (1991)); and 3) maize promoter, Zag2 (“Identification and molecular characterization of ZAG1, the maize homolog of the Arabidopsis floral homeotic gene AGAMOUS”, Schmidt, R. J. et al., Plant Cell 5(7):729-737 (1993); “Structural characterization, chromosomal localization and phylogenetic evaluation of two pairs of AGAMOUS-like MADS-box genes from maize”, Theissen et al. Gene 156(2):155-166 (1995); NCBI GenBank Accession No. X80206)). Zag2 transcripts can be detected 5 days prior to pollination to 7 to 8 days after pollination (“DAP”), and directs expression in the carpel of developing female inflorescences and CimI which is specific to the nucleus of developing maize kernels. CimI transcript is detected 4 to 5 days before pollination to 6 to 8 DAP. Other useful promoters include any promoter which can be derived from a gene whose expression is maternally associated with developing female florets.

Promoters for use also include the following: Zm-GOS2 (maize promoter for “Gene from Oryza sativa”, US publication number US2012/0110700 Sb-RCC (Sorghum promoter for Root Cortical Cell delineating protein, root specific expression), Zm-ADF4 (U.S. Pat. No. 7,902,428; Maize promoter for Actin Depolymerizing Factor), Zm-FTM1 (U.S. Pat. No. 7,842,851; maize promoter for Floral transition MADSs) promoters.

Additional promoters for regulating the expression of the nucleotide sequences in plants are stalk-specific promoters. Such stalk-specific promoters include the alfalfa S2A promoter (GenBank Accession No. EF030816; Abrahams et al., Plant Mol. Biol. 27:513-528 (1995)) and S2B promoter (GenBank Accession No. EF030817) and the like, herein incorporated by reference.

Promoters may be derived in their entirety from a native gene, or be composed of different elements derived from different promoters found in nature, or even comprise synthetic DNA segments.

In the present disclosure, the at least one regulatory element may be an endogenous promoter operably linked to at least one enhancer element; e.g., a 35S, nos or ocs enhancer element.

Promoters for use may include: RIP2, mLIP15, ZmCOR1, Rab17, CaMV 35S, RD29A, B22E, Zag2, SAM synthetase, ubiquitin, CaMV 19S, nos, Adh, sucrose synthase, R-allele, the vascular tissue preferred promoters S2A (Genbank accession number EF030816) and S2B (Genbank accession number EF030817), and the constitutive promoter GOS2 from Zea mays. Other promoters include root preferred promoters, such as the maize NAS2 promoter, the maize Cyclo promoter (US 2006/0156439, published Jul. 13, 2006), the maize ROOTMET2 promoter (WO05063998, published Jul. 14, 2005), the CR1BIO promoter (WO06055487, published May 26, 2006), the CRWAQ81 (WO05035770, published Apr. 21, 2005) and the maize ZRP2.47 promoter (NCBI accession number: U38790; GI No. 1063664),

Polynucleotides encoding a transcript of a line-specific or cluster-specific gene identified by any of the methods disclosed herein or recombinant DNA constructs of the present disclosure may also include other regulatory sequences, including but not limited to, translation leader sequences, introns, and polyadenylation recognition sequences. In the present disclosure, a polynucleotide encoding a transcript of a line-specific or cluster-specific gene identified by any of the methods disclosed herein or a recombinant DNA construct may further comprises an enhancer or silencer.

The promoters disclosed herein may be used with their own introns, or with any heterologous introns to drive expression of the transgene.

An intron sequence can be added to the 5′ untranslated region, the protein-coding region or the 3′ untranslated region to increase the amount of the mature message that accumulates in the cytosol. Inclusion of a spliceable intron in the transcription unit in both plant and animal expression constructs has been shown to increase gene expression at both the mRNA and protein levels up to 1000-fold. Buchman and Berg, Mol. Cell Biol. 8:4395-4405 (1988); Callis et al., Genes Dev. 1:1183-1200 (1987).

“Transcription terminator”, “termination sequences”, or “terminator” refer to DNA sequences located downstream of a protein-coding sequence, including polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht, I. L., et al., Plant Cell 1:671-680 (1989). A polynucleotide sequence with “terminator activity” generally refers to a polynucleotide sequence that, when operably linked to the 3′ end of a second polynucleotide sequence that is to be expressed, is capable of terminating transcription from the second polynucleotide sequence and facilitating efficient 3′ end processing of the messenger RNA resulting in addition of poly A tail. Transcription termination is the process by which RNA synthesis by RNA polymerase is stopped and both the processed messenger RNA and the enzyme are released from the DNA template.

Improper termination of an RNA transcript can affect the stability of the RNA, and hence can affect protein expression. Variability of transgene expression is sometimes attributed to variability of termination efficiency (Bieri et al (2002) Molecular Breeding 10: 107-117).

Examples of terminators for use include, but are not limited to, PinII terminator, SB-GKAF terminator (U.S. Appln. No. 61/514,055), Actin terminator, Os-Actin terminator, Ubi terminator, Sb-Ubi terminator, Os-Ubi terminator.

Any plant can be selected for the identification of regulatory sequences to be used in recombinant DNA constructs and other compositions (e.g. transgenic plants, seeds and cells) and methods of the present disclosure. Examples of suitable plants for the isolation of genes and regulatory sequences for compositions and methods of the present disclosure would include but are not limited to alfalfa, apple, apricot, Arabidopsis, artichoke, arugula, asparagus, avocado, banana, barley, beans, beet, blackberry, blueberry, broccoli, brussels sprouts, cabbage, canola, cantaloupe, carrot, cassava, castorbean, cauliflower, celery, cherry, chicory, cilantro, citrus, clementines, clover, coconut, coffee, corn, cotton, cranberry, cucumber, Douglas fir, eggplant, endive, escarole, eucalyptus, fennel, figs, garlic, gourd, grape, grapefruit, honey dew, jicama, kiwifruit, lettuce, leeks, lemon, lime, Loblolly pine, linseed, mango, melon, mushroom, nectarine, nut, oat, oil palm, oil seed rape, okra, olive, onion, orange, an ornamental plant, palm, papaya, parsley, parsnip, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, sorghum, Southern pine, soybean, spinach, squash, strawberry, sugarbeet, sugarcane, sunflower, sweet potato, sweetgum, switchgrass, tangerine, tea, tobacco, tomato, triticale, turf, turnip, a vine, watermelon, wheat, yams, and zucchini.

The polynucleotide encoding a transcript of a line-specific or cluster-specific gene identified by any of the methods disclosed herein or the recombinant DNA construct may be stably integrated into the genome of the plant. The plant may be used in the methods described herein.

### Transformation:

A method for transforming a cell (or microorganism) comprising transforming a cell (or microorganism) with any of the isolated polynucleotides encoding a transcript of a line-specific or cluster-specific gene identified by any of the methods disclosed herein or recombinant DNA constructs of the present disclosure. The cell (or microorganism) transformed by this method is also included. In the present disclosure, the cell may be eukaryotic, e.g., a yeast, insect or plant cell, or prokaryotic, e.g., a bacterial cell. The microorganism may be Agrobacterium, e.g. Agrobacterium tumefaciens or Agrobacterium rhizogenes.

A method for producing a transgenic plant comprising transforming a plant cell with any of the isolated polynucleotides encoding a transcript of a line-specific or cluster-specific gene identified by any of the methods disclosed herein or recombinant DNA constructs (including suppression DNA constructs) of the present disclosure and regenerating a transgenic plant from the transformed plant cell. The disclosure is also directed to the transgenic plant produced by this method, and transgenic seed obtained from this transgenic plant. The transgenic plant obtained by this method may be used in other methods of the present disclosure.

A method for isolating a polypeptide of the disclosure from a cell or culture medium of the cell, wherein the cell comprises a polynucleotide encoding a transcript of a line-specific or cluster-specific gene identified by any of the methods disclosed herein or a recombinant DNA construct comprising a polynucleotide of the disclosure operably linked to at least one heterologous regulatory sequence, and wherein the transformed host cell is grown under conditions that are suitable for expression of the polynucleotide recombinant DNA construct.

In any of the methods of the present disclosure, alternatives exist for introducing into a regenerable plant cell a recombinant DNA construct comprising a polynucleotide operably linked to at least one regulatory sequence. For example, one may introduce into a regenerable plant cell a regulatory sequence (such as one or more enhancers, optionally as part of a transposable element), and then screen for an event in which the regulatory sequence is operably linked to an endogenous gene encoding a polypeptide of the instant disclosure.

The introduction of the polynucleotides or recombinant DNA constructs of the present disclosure into plants may be carried out by any suitable technique, including but not limited to direct DNA uptake, chemical treatment, electroporation, microinjection, cell fusion, infection, vector-mediated DNA transfer, bombardment, or Agrobacterium-mediated transformation. Techniques for plant transformation and regeneration have been described in International Patent Publication WO 2009/006276, the contents of which are herein incorporated by reference.

The development or regeneration of plants containing the foreign, exogenous isolated nucleic acid fragment that encodes a protein of interest is well known in the art. The regenerated plants may be self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present disclosure containing a desired polypeptide is cultivated using methods well known to one skilled in the art.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

Complete sequences and figures for vectors described herein (e.g., pHSbarENDs2, pDONR™/Zeo, pDONR™221, pBC-yellow, PHP27840, PHP23236, PHP10523, PHP23235 and PHP28647) are given in PCT Publication No. WO/2012/058528, the contents of which are herein incorporated by reference. The present disclosure also includes the following:

1. A plant (for example, a maize, rice or soybean plant) comprising in its genome a polynucleotide encoding a transcript of a line-specific or cluster-specific gene identified by any of the methods disclosed herein or a recombinant DNA construct comprising a polynucleotide encoding a transcript of a line-specific or cluster-specific gene identified by any of the methods disclosed herein, wherein the polynucleotide is operably linked to at least one heterologous regulatory sequence, and wherein said plant exhibits at least one phenotype selected from the group consisting of increased yield, increased productivity and increased stress resistance, when compared to a control plant not comprising said polynucleotide encoding a transcript of a line-specific or cluster-specific gene or recombinant DNA construct. The plant may further exhibit an alteration of at least one agronomic characteristic when compared to the control plant.

2. Any progeny of the plants described herein, any seeds of the plants described herein, any seeds of progeny of the plants described herein, and cells from any of the above plants described herein and progeny thereof.

In the present disclosure, the plant may exhibit alteration of at least one agronomic characteristic selected from the group consisting of: abiotic stress tolerance, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, leaf number, tiller number, growth rate, first pollen shed time, silk length, first silk emergence time, anthesis silking interval (ASI), stalk diameter, root architecture, staygreen, relative water content, water use, water use efficiency, dry weight of either main plant, tillers, primary ear, main plant and tillers or cobs; rows of kernels, total plant weight, kernel weight, kernel number, salt tolerance, chlorophyll content, flavonol content, number of yellow leaves, early seedling vigor and seedling emergence under low temperature stress. These agronomic characteristics maybe measured at any stage of the plant development. One or more of these agronomic characteristics may be measured under stress or non-stress conditions, and may show alteration on perturbation of expression of at least one line-specific gene and/or at least one cluster-specific gene.

In the present disclosure, the polynucleotide encoding a transcript of a line-specific or cluster-specific gene identified by any of the methods disclosed herein or the recombinant DNA construct (or suppression DNA construct) may comprise at least a promoter functional in a plant as a regulatory sequence.

1. Progeny of a transformed plant which is hemizygous with respect to a polynucleotide encoding a transcript of a line-specific or cluster-specific gene identified by any of the methods disclosed herein or recombinant DNA construct (or suppression DNA construct), such that the progeny are segregating into plants either comprising or not comprising the polynucleotide or the recombinant DNA construct (or suppression DNA construct): the progeny comprising the polynucleotide or recombinant DNA construct (or suppression DNA construct) would be typically measured relative to the progeny not comprising the polynucleotide or recombinant DNA construct (or suppression DNA construct) (i.e., the progeny not comprising the recombinant DNA construct (or the suppression DNA construct) is the control or reference plant).

2. Introgression of a polynucleotide encoding a transcript of a line-specific or cluster-specific gene identified by any of the methods disclosed herein or recombinant DNA construct (or suppression DNA construct) into an inbred line, such as in maize, or into a variety, such as in soybean: the introgressed line would typically be measured relative to the parent inbred or variety line (i.e., the parent inbred or variety line is the control or reference plant).

3. Two hybrid lines, where the first hybrid line is produced from two parent inbred lines, and the second hybrid line is produced from the same two parent inbred lines except that one of the parent inbred lines contains a polynucleotide encoding a transcript of a line-specific or cluster-specific gene identified by any of the methods disclosed herein or recombinant DNA construct (or suppression DNA construct): the second hybrid line would typically be measured relative to the first hybrid line (i.e., the first hybrid line is the control or reference plant).

4. A plant comprising a polynucleotide encoding a transcript of a line-specific or cluster-specific gene identified by any of the methods disclosed herein or recombinant DNA construct (or suppression DNA construct): the plant may be assessed or measured relative to a control plant not comprising the polynucleotide or recombinant DNA construct (or suppression DNA construct) but otherwise having a comparable genetic background to the plant (e.g., sharing at least 90%, 91%, 92%, 93%, 94%, 95%, 96%, 97%, 98%, 99%, or 100% sequence identity of nuclear genetic material compared to the plant comprising the recombinant DNA construct (or suppression DNA construct)). There are many laboratory-based techniques available for the analysis, comparison and characterization of plant genetic backgrounds; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLP®s), and Simple Sequence Repeats (SSRs) which are also referred to as Microsatellites.

Furthermore, one of ordinary skill in the art would readily recognize that a suitable control or reference plant to be utilized when assessing or measuring an agronomic characteristic or phenotype of a transgenic plant would not include a plant that had been previously selected, via mutagenesis or transformation, for the desired agronomic characteristic or phenotype.

### EXAMPLES

The present disclosure is further illustrated in the following Examples, in which parts and percentages are by weight and degrees are Celsius, unless otherwise stated. It should be understood that these Examples of the present disclosure are given by way of illustration only. From the above discussion and these Examples, one skilled in the art can ascertain the essential characteristics of this disclosure, and without departing from the spirit and scope thereof, can make various changes and modifications of the disclosure to adapt it to various usages and conditions. Thus, various modifications of the disclosure in addition to those shown and described herein will be apparent to those skilled in the art from the foregoing description. Such modifications are also intended to fall within the scope of the appended claims.

### Example 1

### Identification of a Line-Specific Gene

### Arabidopsis NUE Data Set:

Arabidopsis gene expression data set was used for identifying a line-specific gene/marker (LSM) from a set of 48 transgenic plants. Data from two tissues was collected: root and shoot.

Transcriptomics data for the 48 plants (with perturbation of 48 different transgenes) and one wild-type (control) plant was collected using Agilent-032829(8×64 chip type) Microarray technology which can include the expression from ˜60000 probes.

The 48 transgenes had been validated to confer low nitrogen stress tolerance, or increase nitrogen uptake or increase root mass in Arabidopsis plants (US patent publication Nos. US20090011516, US20160040181, and US20110138501).

Out of these 48 transgenes, 46 transgenes were overexpressed and two transgenes were downregulated. The two that were downregulated were two mutant lines that create a full length mRNA but a mutant protein. These plant samples (48 transgenic samples and 1 control sample) were subjected to low nitrogen stress conditions (0.5 mM KNO₃) which is less than the normal nitrogen condition (4 mM KNO₃). The plants for collecting samples for the low nitrogen condition were grown in nitrogen plates with low nitrogen conditions (0.5 mM KNO₃).

For each of the 48 transgenic samples 3-4 replicates of each transgenic plant were run and for the WT samples total 80 replicates were run. The total number of the samples run were 506 samples

The transcriptomics expression matrix that was used for the study contained expression data from ˜60000 probes for 506 samples (48 transgenes+1 control). These ˜60000 probes mapped to 31000 Arabidopsis genes.

### Computational Strategy for Identifying Line-Specific Genes or Line-Specific Markers (LSMs)(for one Transgene):

### Filtering the Data Set:

The transcriptomics data set that Agilent Microarray technology generates is not normalized across arrays. The data set was first normalized across all the arrays using R package limma.

The normalized data set was then used for further analysis. All the 60000 probes were checked for their differential expression in the transgenic plant with perturbed expression of the particular transgene for which LSMs were to be identified, with respect to the control WT samples.

One of the 48 transgenes used in this pool was At1g07630 (PP2C), which was overexpressed by cloning it downstream of the CaMV 35S promoter. LSMs for the transgene 35S:AT1G07630 (PP2C) were identified in both root and shoot tissue samples separately. At1g07630 (PP2C) has been shown to be responsible for altering root architecture under high nitrogen conditions (60 mM KNO₃), and also has been shown to confer low nitrogen stress tolerance (US Patent Publication No. 2011/0138501).

The differential expression analysis was run for the 35S:AT1G07630 with respect to the WT samples.

The p-value cutoff was used to filter off the list of genes that have differential expression compared to the WT samples. The p-value cutoff used in this case was <=0.1. Using this cut off value, the number of genes that were differentially expressed in 35S:AT1G07630 plants as compared to WT in root tissue samples only was 6302 and in shoot tissue only was 9380. Data from only these genes was used for identification of the LSMs.

### Running Random Forest Algorithm:

The data from the above two filtered lists was used from root and shoot tissue samples and then random Forest algorithm in supervised mode was run to generate the list of genes which were ranked according to their ability to distinguish samples from 35S:AT1G07630 plants from the rest of the transgenic samples.

While running random forest algorithm, the WT samples were not used. Only the transgenic samples were used. In this case the LSMs were identified for the transgene AT1G07630 that could distinguish the At1g07630 overexpressing plants from the rest of the transgenic plant samples.

Two classes of samples were made: YES class that included 4 replicates of the samples overexpressing the transgene of our interest, AT1G07630 and NO class that included samples from rest of the transgenes (47×4). Because there was so much unbalanced data set, “strata” parameter of random forest was set to 0.75. This parameter was used to ensure that at all iterations of random forest only 0.75 of the samples were taken into account from both the classes. From the total probe set in expression data, randomForest selects features randomly(sqrt(total probe)) for generating decision trees in forest. The number of probes selected by randomForest can be set up using the “mtry” parameter. For this analysis the “mtry” parameter was set as 0.8*sqrt(60000).

This information of YES and NO classes was provided to the random Forest algorithm in supervised mode. 20000 trees were run in this example. So, the genes were ranked according to the importance value given by the random Forest algorithm which is based on the ability of these genes to distinguish samples from YES and NO class. The better the importance value given to the gene, the more was the confidence on the gene to be called as a line-specific gene or a line-specific marker for the transgene AT1G07630. The randomForest algorithm was run on the filtered set for root and shoots tissues separately.

### Generating Final List of LSMs:

Top 5-20 LSMs which are ranked according to the importance values from random Forest are taken to be LSMs for the transgene_AT1G07630 (referred as D3 in Table 2) from both root and shoot tissue data separately.

Tissue agnostic LSMs were the ones that are finally listed for testing. These tissue agnostic LSMs were called as tissue ubiquitous LSMs. If the LSM had either positive or negative fold-change and p-value<=0.1 in both root and shoot tissues as compared to the control samples, then these LSMs were called as ubiquitous LSMs. These lists of LSMs were further tested for their phenotypes in diverse assays in control environment conditions.

LSMs were identified for the transgene AT1G07630 overexpressed in Arabidopsis plants that came out to be differentially expressed in both root and shoot tissue, and showed alteration of root architecture when overexpressed in Arabidopsis plants.

Four LSM candidates of AT1G07630 (D3 in Table 2) were chosen for testing in Arabidopsis to determine if any LSM candidate showed a phenotype similar to overexpression of AT1G07630, the primary gene line (D3 in Table 2). Similar to AT1G07630, all LSM candidates were overexpressed in Arabidopsis with the CaMV 35S promoter. LSM1 passed both the low nitrogen plate assay and root architecture assay similar to AT1G07630. Thus, this LSM1 was nominated for testing in maize.

LSMs for other transgenes from these 48 transgenes were also identified, plants overexpressing a subset of these LSMs showed the same agronomic characteristics of increased nitrogen uptake, altered root architecture, or increased nitrogen stress tolerance as their respective primary gene line when compared to control plants. Table 1 summarizes the results from testing LSMs derived from primary lines that originally passed the low nitrogen (LN) assay, as described for phase 3 screen in US Patent Application Publication No. 20160040181. Overall, 39% of the LSMs tested in this assay were deemed as validated, resulting in 12 out of 17 primary lines having at least 1 LSM validate. Table 2 summarizes the results from testing LSMs derived from primary lines that originally passed the root architecture (RA) assay. Nine of the 11 primary lines had at least 1 LSM deemed as validated according to the description in US Patent Publication No. 2011/0138501. Overall, 34% of the LSMs tested for these 11 primary lines validated in this assay.

TABLE 1
Results from LSM testing in low nitrogen assay

| | Primary | | |
|---|---|---|---|
| Arabidopsis | Gene/Driver | | #LSM |
| Assay | Code | #LSM Tested | Validated |
| LN | D1 | 1 | 1 |
| LN | D2 | 3 | 0 |
| LN | D3 | 3 | 0 |
| LN | D4 | 7 | 4 |
| LN | D5 | 7 | 1 |
| LN | D6 | 3 | 0 |
| LN | D7 | 1 | 1 |
| LN | D8 | 2 | 1 |
| LN | D9 | 6 | 3 |
| LN | D10 | 2 | 1 |
| LN | D11 | 5 | 2 |
| LN | D12 | 1 | 0 |
| LN | D13 | 4 | 4 |
| LN | D14 | 3 | 0 |
| LN | D15 | 5 | 2 |
| LN | D16 | 9 | 4 |
| LN | D17 | 2 | 1 |
| Total | 17 | 64 | 25 |

TABLE 2
Results from LSM testing in root architecture assay

| | Primary | | |
|---|---|---|---|
| Arabidopsis | Gene/Driver | | |
| Assay | Code | # LSM Tested | # LSM Validated |
| RA | D1 | 2 | 1 |
| RA | D2 | 6 | 2 |
| RA | D3 | 4 | 1 |
| RA | D4 | 2 | 1 |
| RA | D5 | 4 | 1 |
| RA | D6 | 2 | 0 |
| RA | D7 | 5 | 2 |
| RA | D8 | 3 | 2 |
| RA | D9 | 1 | 0 |
| RA | D10 | 1 | 1 |
| RA | D11 | 2 | 1 |
| | 11 | 32 | 12 |

### Example 2

### Identification of a Cluster-Specific Gene or Cluster-Specific Marker Data Set

The data set described in Example 1 was used for identifying cluster-specific gene or cluster-specific marker (CSM)

### Computational Strategy for Identifying CSMs.

### Filtering the Data Set:

The strategy used for filtering the data set was the same as described in Example 1, for identifying line-specific gene.

### Running Random Forest Algorithm:

Random forest algorithm in supervised manner was run for the samples from 48 different transgenes in same way as described in Example 1. The top 100 genes from each of the 48 transgenes were ranked based on the importance value criteria from random forest method.

These top 100 genes, which were ranked using importance values from random forest algorithm, were taken for further analysis. Then gene expression data from the top 100 genes from all the 48 transgenes samples was taken separately for root and shoot tissues for further analysis.

The gene expression data from the top genes was then used as an input to run unsupervised random forest algorithm, from which the proximity values for the 48 different transgenes samples was calculated. The proximity matrix was a square similarity matrix. This similarity matrix was converted into a distance matrix which is defined as: distance matrix=1−proximity matrix.

This distance matrix was given as input to the Hclust program from R base package to generate clusters from these 48 different transgenes for root and shoot tissues separately. The Hclust program uses “ward” method to generate the clusters of the 48 transgenes in which WT samples are also included.

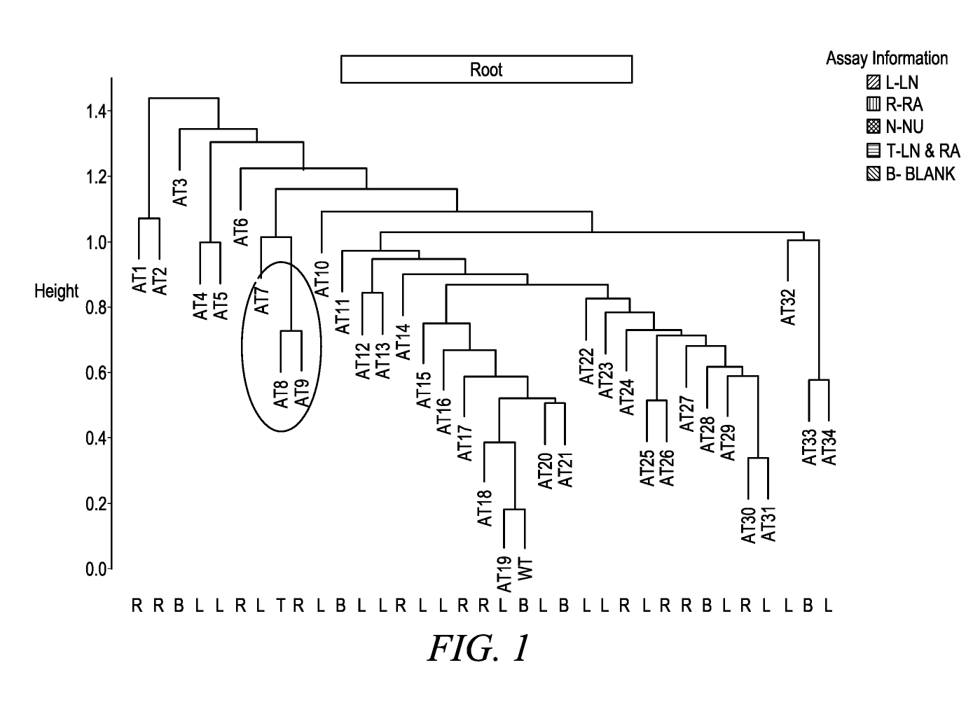

The cluster of the transgenes can be defined as, a cluster that has minimum of two transgenes clustered together in the last node of the cluster tree. In this example the cluster shown in FIG. 1 is from root tissue in which the plants were subjected to low nitrogen condition.

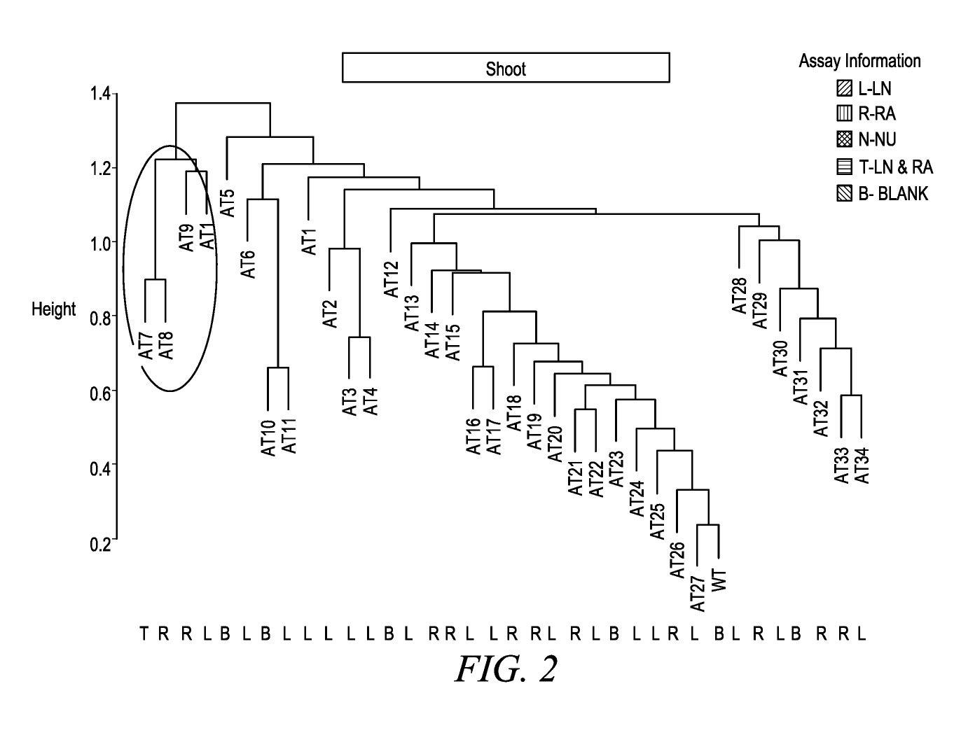

As shown in FIG. 1, in this case, the three transgene cluster (marked with the oval) is taken as it is a robust cluster which also comes in shoot tissue as well (as seen in FIG. 2).

As shown in FIG. 2, this is a cluster that comes under root and shoot tissue as well, so this cluster (marked with the oval) was picked in this case. In the case mentioned above, the clusters (marked with the oval) belong to root and shoot tissue both and transgenes in this cluster have shown positive phenotype in similar assays.

### Generating Final List of CSMs:

The gene expression data from top 100 genes was picked from all the three transgenes that belonged to the cluster shown above in the oval for further analysis.

The gene expression data from union of these top 100 genes (˜300) from these three transgenes was checked for their expression in samples that belonged to these three transgenes in both root and shoot.

The genes that showed high expression as compared to the WT samples in at least 80% of the transgenes in this cluster were further checked for their expression in rest of the transgenic samples that does not belong to this cluster. If these genes also, have expression in less 20% of the rest of the transgenes (not included in this cluster) then these genes were called as CSMs. The opposite scenario of lower expression in the chosen cluster and higher expression in rest is also permissible.

### Example 3

### Clustering Plants Based on other Criteria

To identify a cluster-specific gene from a cluster of plants belonging to a plurality of plants, other criteria may be used. Plants can be clustered on the basis of:

### Clustering Plants Based on Sequence Similarity of Primary Genes:

Transgenic plant lines can be clustered based on pairwise sequence similarity of all the transgenes. Once a cluster is derived based on hierarchical cluster or other commonly used clustering techniques, one can look for genes using Machine Learning techniques having unique expression pattern in a chosen cluster compared to others. These genes will be the cluster specific marker of the chosen cluster.

### Clustering Plants Based on Phenotype or Agronomic Characteristics:

Each transgenic line can be phenotypically characterized by multiple different assays. rtPhenotype scores can be used as quantitative values to deduce similarity between transgenic lines, which further like the previous case, can be used for clustering. CSM can be derived from clusters as described above.

Clusters of those transgenes that has shown positive phenotype in the similar or same assays can be made, for example, in the example given here, the cluster of AT2, AT3 and AT4 transgene can be picked that belong to the same assay Low Nitrogen stress tolerance).

The clusters of those transgenes can also be picked if they are clustering together with a transgene having a phenotype of interest from prior knowledge.

Clusters of plants from a plurality of plants can also be made when all the plants exhibit perturbation of expression of the same primary gene, but exhibit different phenotypes. Different plant events obtained by overexpressing or downregulating the same transgene many times exhibit different phenotypes such as different yields. Clusters of different plant events can be made based on their agronomic characteristics such as yield.

### Example 4

### Yield Analysis of Maize Lines with the Line-Specific Gene or Cluster-Specific Gene

A recombinant DNA construct containing an Arabidopsis line-specific gene or cluster-specific gene can be introduced into an elite maize inbred line either by direct transformation or introgression from a separately transformed line.

Transgenic plants either inbred or hybrid, can undergo more vigorous field-based experiments to study yield enhancement and/or stability under well-watered, low nitrogen and water-limiting conditions.

Transgenic Event Analysis from Field Plots for Drought Tolerance

Subsequent yield analysis can be done to determine whether plants that contain the validated Arabidopsis line-specific gene or cluster-specific gene have an improvement in yield performance under water-limiting conditions, when compared to the control plants that do not contain the validated Arabidopsis line-specific gene or cluster-specific gene. Specifically, drought conditions can be imposed during the flowering and/or grain fill period for plants that contain the validated Arabidopsis lead gene and the control plants. Reduction in yield can be measured for both. Plants containing the validated Arabidopsis line-specific gene or cluster-specific gene have less yield loss relative to the control plants, for example, at least 25%, at least 20%, at least 15%, at least 10% or at least 5% less yield loss.

The above method may be used to select transgenic plants with increased yield, under water-limiting conditions and/or well-watered conditions, when compared to a control plant not comprising said recombinant DNA construct. Plants containing the validated Arabidopsis line-specific gene or cluster-specific gene may have increased yield, under water-limiting conditions and/or well-watered conditions, relative to the control plants, for example, at least 5%, at least 10%, at least 15%, at least 20% or at least 25% increased yield.

Transgenic Event Analysis from Field Plots under Various Nitrogen Conditions

Subsequent yield analysis can be done to determine whether plants that contain the validated Arabidopsis line-specific gene or cluster-specific gene have an improvement in yield performance under various nitrogen conditions. Plants containing the validated Arabidopsis line-specific gene or cluster-specific gene may have less yield loss relative to the control plants, for example, under various nitrogen conditions, optimized or low nitrogen. The expectation is that some validated LSMs or CSMs from the Arabidopsis assays may show a significant improvement for yield or yield-related traits in maize under these nitrogen conditions. One of skill will recognize the appropriate promoter to use to modulate the level/activity of a gene in the plant to achieve the desired phenotypic effect.

In general, transgenic events may be molecular characterized for transgene copy number and expression by PCR. Events containing single copy of transgene with detectable transgene expression may be advanced for field testing. Test cross/hybrid seeds are produced and tested in field in multi-years/locations/replications experiments both in normal and low N fields. Transgenic events are evaluated in field plots where yield is limited by reducing fertilizer application by 30% or more. Statistically significant improvements in yield, yield components or other agronomic traits between transgenic and non-transgenic plants in these reduced or normal nitrogen fertility plots are used to assess the efficacy of transgene expression. The constructs with multiple events showing significant improvements (when compared to nulls) in yield or its components in multiple locations are advanced for further testing.

LSM1 identified from AT1G07630 (D3 in Table 2) primary gene (driver) line was overexpressed using a maize constitutive promoter and transformed into maize. Seven transgenic events were field tested at 5 optimal locations. Yield data were collected in all locations with 3-4 replicates per location. Yield data from multi-location are shown in Table 3 as percentage of difference compared to the control. In Table 3, five transgenic events (A, D-G) overexpressing LSM1 with a constitutive promoter resulted in a statistically significant yield increase of 1.79-4.8% compared to the control under normal nitrogen conditions. Top three events (E-G) showed yield increase of 3.5-4.8% compared to the control. The increase in yield in Event B and Event C is not statistically significant. Transgenic events may have different expression levels of the transgene or different protein levels.

After 2 years of field testing, two (Event E and F) out of seven events maintained a significant increase in yield under various nitrogen conditions.

TABLE 3
1^{st} year yield data from LSM1 transgenic from multi-locations.

| Event | Yield (%) |
|---|---|
| A | 2.13 |
| B | 1.18 |
| C | 1.57 |
| D | 1.79 |
| E | 4.80 |
| F | 3.50 |
| G | 3.76 |

### Example 5

### Identification of a Line-Specific Gene

### Data Set for Maize:

In the transcriptomics expression matrix that was used for the study contained expression data (read count data) from ˜100,000 transcripts from which, after removing low quality transcripts, about ˜65,000 transcripts expression data were used for LSM analysis. The data was collected for total 1411 samples (25 transgenes+1 control) from three tissues root, leaf and ear in four developmental stages—v14, v16, v18 and r01 under drought stress and unstressed condition. 3-4 biological replicates were sampled for each transgenic×stage×tissue×treatment condition.

Maize gene expression data set was also used for identifying a line-specific gene/marker (LSM) from a set of 25 transgenic lines. Data from three tissues were collected: root, shoot and immature ear from 4 developmental stages were collected.

Transcriptomics data for the 25 transgenic lines (with perturbation of 22 different transgenes) and control plants (wild-type and bulk null) was collected using Illumina RNA-seq technology which can include the expression from ˜100,000 transcripts. After low quality transcripts were removed, expression data from ˜65,000 transcripts were used for the line-specific marker analysis as described in Example 1.

Out of these 25 transgenic lines, 24 transgenes were overexpressed and one transgene was downregulated. These plant samples (transgenic samples and 1 control sample) were subjected to stress conditions (low nitrogen or drought) and unstressed conditions in field testing locations.

For each of the 25 transgenic lines 3-4 replicates of each transgenic plant were collected along with WT and bulk null samples. The total number of the samples run were 1411 samples.

These lists of LSMs will be further tested for their phenotypes in diverse assays in greenhouse environment and in field conditions.

Transgenic events may be molecular characterized for transgene copy number and expression by PCR. Events containing single copy of transgene with detectable transgene expression may be advanced for field testing. Test cross/hybrid seeds are produced and tested in field in multi-years/locations/replications experiments both in normal and low N fields. Transgenic events are evaluated in field plots where yield is limited by reducing fertilizer application by 30% or more. Statistically significant improvements in yield, yield components or other agronomic traits between transgenic and non-transgenic plants in these reduced or normal nitrogen fertility plots are used to assess the efficacy of transgene expression. The constructs with multiple events showing significant improvements (when compared to nulls) in yield or its components in multiple locations were are advanced for further testing.

1. A method of identifying at least one line-specific gene from a plurality of plants, wherein all plants in the plurality of plants exhibit alteration in at least one first agronomic characteristic, and wherein the alteration in the at least one first agronomic characteristic in each plant in the plurality of plants is due to perturbation of expression of a different primary gene, when compared to a control plant that does not show the alteration in the at least one first agronomic characteristic, the method comprising the steps of:
(a) analyzing gene expression in each plant in the plurality of plants to identify genes that show perturbation of expression when compared to a control plant;
(b) comparing gene expression data from a first plant in the plurality of plants to gene expression data from other plants in the plurality of plants to identify at least one line-specific gene from the first plant, wherein the at least one line-specific gene shows perturbation of expression in the first plant, and wherein the at least one line-specific gene from the first plant does not show the same perturbation of expression in any of the other plants in the plurality of plants.

2. The method of claim 1, wherein the method further comprises the step of selecting a line-specific gene, wherein the line-specific gene confers upon a plant an alteration in the at least one first agronomic characteristic, wherein the plant shows a perturbation in expression of the line-specific gene when compared to a control plant.

3. The method of claim 1, wherein the perturbation in the line-specific gene can be used as a marker for the first plant to distinguish the first plant from the other plants in the plurality of plants.

4. The method of claim 1, wherein the perturbation of expression of the primary gene is overexpression.

5. The method of claim 1, wherein the perturbation of expression of the primary gene is downregulation.

6. The method of claim 1, wherein at least one of the steps of the method is done computationally.

7. The method of claim 1, wherein step (b) is done by using a machine learning algorithm.

8. The method of claim 1, wherein the order of partial correlation between said first gene with perturbed expression in the first plant and said line-specific gene identified from the first plant in the plurality of plants is not more than two.

9. A method of identifying at least one cluster specific gene from a plurality of plants, wherein all plants in the plurality of plants exhibit an alteration in at least one first agronomic characteristic, the method comprising the steps of:
(a) identifying at least one first cluster of plants and at least one second cluster of plants from the plurality of plants, wherein clustering is done on the basis of criteria selected from the group consisting of:
(i) alteration in at least one second agronomic characteristic in all the plants of a cluster;
(ii) similarity in gene expression profile between the plants of a cluster as determined by the distance metric with a cluster bootstrap confidence value of at least 50%;
(iii) perturbed expression of polypeptides from the same gene family in all plants from the same cluster;
(b) analyzing gene expression in plants from the at least one first cluster of plants and the at least one second cluster of plants;
(c) comparing the gene expression data from the at least one first cluster of plants to the gene expression data from the at least one second cluster of plants;
(d) identifying at least one cluster-specific gene that is perturbed in at least 80% of the plants from the at least one first cluster of plants, and perturbed in not more than 20% of the plants from the at least one second cluster of plants.

10. The method of claim 9, wherein the alteration in the at least one first agronomic characteristic in each plant in the plurality of plants is due to perturbation of expression of a different gene.

11. The method of claim 9, wherein the alteration in the at least one first agronomic characteristic in each plant in the plurality of plants is due to perturbation of expression of the same gene.

12. The method of claim 9, wherein it further comprises the step of selecting a cluster-specific gene, wherein the cluster-specific gene confers upon a plant an alteration in the at least one first agronomic characteristic, wherein the plant shows a perturbation in expression of the cluster-specific gene when compared to a control plant.

13. The method of claim 9, wherein at least one of the steps of the method is done computationally.

14. The method of claim 9, wherein at least one of the steps of the method is done by using a machine learning algorithm.

15. The method of claim 1, wherein each plant in the plurality of plants comprises a recombinant construct comprising a polynucleotide sequence that comprises the coding region of the primary gene operably linked to at least one heterologous regulatory element.

16. The method of claim 1, wherein the step for analyzing gene expression data is done in specific tissues.

17. The method of claim 1, wherein said line-specific gene identified from the plurality of plants shows perturbation of expression in all the tissues analyzed for gene expression.

18. The method of claim 9, wherein the bootstrap confidence value for the plants in the same cluster is at least 60%.

19. The method of claim 9, wherein the expression of the cluster specific gene identified in step (d) is perturbed in not more than 10% of the plants from the at least one second cluster of plants.

20. The method of claim 1, wherein the plurality of plants comprises of at least two plants.

21. The method of claim 1, wherein the plurality of plants comprises of at least 10 plants.

22. The method of claim 1, wherein all plants in the plurality of plants exhibit alteration in at least one first agronomic characteristic, and wherein said all plants in said plurality of plants exhibit alteration in the same at least one first agronomic characteristic.

23. The method of claim 1, wherein all plants in the plurality of plants exhibit alteration in at least one first agronomic characteristic, and wherein said all plants in said plurality of plants do not exhibit alteration in the same at least one first agronomic characteristic.

24. (canceled)

25. (canceled)

26. (canceled)

27. (canceled)

28. (canceled)

29. The method of claim 1, wherein the line-specific gene is introduced into another plant.

30. The method of claim 29, wherein the wherein the line-specific gene is introduced into another plant using genome editing.

31. The method of claim 9, wherein the cluster-specific gene is introduced into a plant.

32. The method of claim 31, wherein the wherein the cluster-specific gene is introduced into another plant using genome editing.

33. The method of claim 2, wherein the selected line-specific gene encodes a protein variant different from a cognate wild-type protein.

34. The method of claim 2, wherein the selected line-specific gene is tested.

35. The method of claim 12, wherein the selected cluster-specific gene is tested.
